(19) United States
(12) Patent Application Publication
(10) Pub. No.: US 2026/0216075 A1
(43) Pub. Date: Jul. 30, 2026

(54) POLYMERSOMES FOR DELIVERY OF NUCLEIC ACID CARGOES

(71) Applicant: VIANAUTIS BIO LIMITED, Cambridge (GB)

(72) Inventors: Denis CECCHIN, Cambridge (GB); Vigínia M. GOUVEIA, Cambridge (GB); Giuseppe BATTAGLIA, Cambridge (GB)

(21) Appl. No.: 19/145,102

(22) PCT Filed: Jan. 5, 2024
(86) PCT No.: PCT/GB2024/050022

§ 371 Date: Jul. 1, 2025

(30) Foreign Application Priority Data
Jan. 6, 2023 (GB) 2300199.3

(51) Int. Cl. A61K 9/1273; A61K 39/00; C12N 15/88

(52) U.S. Cl. CPC A61K 9/1273; C12N 15/88; A61K 39/00; A61K 2039/55555

(57) ABSTRACT

The present invention relates to the field of polymersomes for the effective delivery of nucleic acid cargoes to target cells within the body. Specifically, the present invention is directed to a cargo-containing polymersome which comprises (a) a polymersome and (b) a cargo encapsulated within the polymersome, wherein said cargo comprises a nucleic acid and a nucleic acid-binding protein. The present invention is further directed to pharmaceutical compositions comprising a plurality of such polymersomes and one or more pharmaceutically acceptable excipients or diluents. The present invention is further directed to uses of such polymersomes and pharmaceutical compositions in medicine, and to vaccines comprising such polymersomes.

### FIELD OF THE INVENTION

The present invention relates to the field of polymersomes for the effective delivery of nucleic acid cargoes to target cells within the body. Specifically, the present invention is directed to a cargo-containing polymersome which comprises (a) a polymersome and (b) a cargo encapsulated within the polymersome, wherein said cargo comprises a nucleic acid and a nucleic acid-binding protein. The present invention is further directed to pharmaceutical compositions comprising a plurality of such polymersomes and one or more pharmaceutically acceptable excipients or diluents. The present invention is further directed to uses of such polymersomes and pharmaceutical compositions in medicine, and to vaccines comprising such polymersomes.

### BACKGROUND TO THE INVENTION

An increasing number of nucleic acid therapeutics and vaccines are being approved for use in the treatment and prevention of disease. Nucleic acids are the subject of much research in these fields, due to the large potential to treat and prevent diseases by targeting genetic blueprints. In contrast to small molecule or biologic therapies which target proteins (e.g. enzymes or receptors), the effects of nucleic acid active agents, particularly DNA, can be long-lasting through gene inhibition, addition, replacement or targeting.

Nucleic acid therapeutics can broadly be categorised into RNA-based therapeutics, DNA-based therapeutics and PNA-based therapeutics.

RNA-based therapeutics include short interfering RNAs (siRNAs), which are double-stranded RNA molecules that are 20-24 nucleotides long (typically 21 nucleotides long) with sequences complementary to a gene's coding sequence to induce degradation of the corresponding messenger RNAs (mRNAs), blocking the translation of mRNA into protein. The first successful proof of concept for siRNA was reported in 2008 (DeVincenzo et al.; Antiviral Res, 2008, 77, 225-231). Another type of RNA therapeutic is microRNA (miRNA), which are single-stranded RNAs about 15-22 nucleotides long, which also function via RNA interference.

The COVID-19 pandemic additionally brought about expedited approval of the first mRNA-based vaccines by regulators including the UK MHRA, the EMA and the US FDA, including Comirnaty® (Pfizer/BioNTech) and Spikevax® (Moderna). Over 600 million doses of mRNA vaccines against SARS-CoV-2 have been administered to date in the USA, and over 700 million doses of mRNA vaccines against SARS-CoV-2 have been administered in the EU (source: https://ourworldindata.org/grapher/covid-vaccine-doses-by-manufacturer; accessed 27 Oct. 2022). These vaccines work by introducing mRNA into cells, which is transcribed to make a protein (e.g. in the case of the coronavirus vaccines, the spike protein of SARS-CoV-2) that induces an immune response in vivo, resulting in the production of antibodies that protect against the viral infection.

There are also several DNA-based therapies currently in development, including T-cell therapies, genome editing and writing, and clustered regularly interspaced short palindromic repeats (CRISPR). DNA therapies can offer advantages over RNA-based therapies, as DNA has e.g. greater stability, an increased durability of expression and lower manufacturing costs than RNA (see e.g. Mirasol; BioPharm International, 2022, 35(4), 16-20).

Antisense oligonucleotides (ASOs) are another category of DNA therapy that have proven useful in medicine: these are short sequences of single-stranded DNA that target a specific, complementary coding or non-coding RNA to induce inhibition of gene expression. The first ASO authorised for human use was fomivirsen, developed in 1998 for the management of cytomegalovirus retinitis (Orr; Curr Opin Mol Ther, 2001, 42, 2646-2651). Subsequently, several other ASOs have also been authorised. The clinical progress of ASOs has however been slow because of the challenges involved with intracellular delivery of these large (compared to small molecules), highly charged molecules within acceptable limits of toxicity (Sridharan and Gogtay; Br J Clin Pharmacol, 2016, 82(3), 659-672).

This is one illustration of a major challenge of nucleic acid therapeutics in general, namely delivery of the therapeutic to the requisite target in vivo. Nucleic acids are very fragile species, and are degraded rapidly in circulation. Nucleic acid active agents must therefore be protected from degradation in a pharmaceutical formulation until they can reach the target site at which they act. Often, the site of action of nucleic acid therapeutics is the nucleus of a cell, so the active agent must be effectively delivered not only to a target cell, but to its nucleus. This challenge is even more acute for DNA-based therapeutics than for RNA-based therapeutics, because DNA must be delivered further into the nucleus to have its effect (Mirasol; BioPharm International, 2022, 35(4), 16-20).

Peptide nucleic acids (PNAs) are another type of nucleic acid that have been the subject of investigation in medical fields. PNA is an artificially synthesized polymer similar to DNA or RNA invented by Peter E. Nielsen (Univ. Copenhagen), Michael Egholm (Univ. Copenhagen), Rolf H. Berg (Risø National Lab), and Ole Buchardt (Univ. Copenhagen) in 1991. PNA's backbone is composed of repeating N-(2-aminoethyl)-glycine units linked by peptide bonds. The various purine and pyrimidine bases are linked to the backbone by a methylene bridge (—CH₂—) and a carbonyl group ((C═O)—).

Delivery systems which are able to protect nucleic acid cargo and deliver it efficiently to its target site of action, leading to a high levels of nucleic acid activity (e.g. gene expression, or gene knockdown), are therefore highly desirable. The present invention addresses this problem and provides a delivery system that improves the efficacy of its nucleic acid cargo.

### SUMMARY OF THE INVENTION

The present inventors have surprisingly discovered that the use of synthetic polymer vesicles (polymersomes) comprising both a nucleic acid cargo and a nucleic acid-binding protein (e.g. a nucleic acid cargo complexed to a nucleic acid-binding protein) provides a particularly effective delivery system for nucleic acid therapeutics to their target site of action (i.e. the nucleus of a target cell).

In particular, such polymersomes can deliver a nucleic acid to cells in a uniform manner, and moreover, the activity of the nucleic acid within the cells is significantly enhanced compared to when the nucleic acid is delivered in a corresponding polymersome which does not also comprise a nucleic acid-binding protein. This therefore represents an improved method of delivering nucleic acid therapeutics in vivo. The polymersomes of this invention are particularly attractive as a versatile delivery system, given that the polymersome vesicles can be adapted (via the choice of ligands presented on the external surface of the vesicles) to be targeted to a variety of different cell types. Thus, the polymersomes of the invention are compatible with a wide variety of different nucleic acid targets which have different biological targets in vivo.

The present invention accordingly provides a cargo-containing microparticle or nanoparticle which comprises: (a) a nanoparticle or a microparticle; and (b) a cargo encapsulated within the nanoparticle or microparticle, wherein said cargo comprises a nucleic acid and a nucleic acid-binding protein. Preferably, the nanoparticle or microparticle is a polymersome. Thus, in a preferred embodiment, the present invention provides a cargo-containing polymersome which comprises: (a) a polymersome; and (b) a cargo encapsulated within the polymersome, wherein said cargo comprises a nucleic acid and a nucleic acid-binding protein.

The present invention further provides a pharmaceutical composition comprising a plurality of the cargo-containing polymersomes of the invention, and one or more pharmaceutically acceptable excipients or diluents.

The present invention further provides a cargo-containing polymersome or a pharmaceutical composition of the invention for use in the treatment of a disease. In preferred embodiments, the disease to be treated is cancer, an infectious disease, a brain disorder, an inflammatory or autoimmune disease, atherosclerosis, ischemic heart disease, a liver disorder, a kidney disorder, a disease associated with ageing, or a genetic disease.

The present invention further provides a method of treating cancer, an infectious disease, a brain disorder, an inflammatory or autoimmune disease, atherosclerosis, ischemic heart disease, a liver disorder, a kidney disorder, a disease associated with ageing, or a genetic disease in a human patient, wherein said method comprises administration of a cargo-containing polymersome or a pharmaceutical composition of the invention to a patient in need thereof.

The present invention further provides the use of a cargo-containing polymersome or a pharmaceutical composition of the invention for the manufacture of a medicament for the treatment of cancer, an infectious disease, a brain disorder, an inflammatory or autoimmune disease, atherosclerosis, ischemic heart disease, a liver disorder, a kidney disorder, a disease associated with ageing, or a genetic disease in a patient.

The present invention further provides a vaccine comprising a cargo-containing polymersome according to the invention, and one or more pharmaceutically acceptable excipients or diluents.

Preferred embodiments of the invention are described in further detail below.

### BRIEF DESCRIPTION OF THE FIGURES

FIG. 1 shows, for polymersomes comprising PEG-PLA as the block copolymer, and free pcDNA3.1NL as the cargo, (a) the particle size distribution by number as measured by dynamic light scattering, (b) the particle size distribution by intensity as measured by dynamic light scattering, (c) a correlogram of the DLS data, and (d) a scanning electron micrograph image of the polymersomes.

FIG. 2 shows, for polymersomes comprising PEG-PLA as the block copolymer, and a cargo comprising pcDNA3.1NL and histone H1, (a) the particle size distribution by number as measured by dynamic light scattering, (b) the particle size distribution by intensity as measured by dynamic light scattering, (c) a correlogram of the DLS data, and (d) a scanning electron micrograph image of the polymersomes.

FIG. 3 shows, for polymersomes comprising PEG-PDPA as the block copolymer, and free pcDNA3.1NL as the cargo, (a) the particle size distribution by number as measured by dynamic light scattering, (b) the particle size distribution by intensity as measured by dynamic light scattering, (c) a correlogram of the DLS data, and (d) a scanning electron micrograph image of the polymersomes.

FIG. 4 shows, for polymersomes comprising PEG-PDPA as the block copolymer, and a cargo comprising pcDNA3.1NL and histone H1, (a) the particle size distribution by number as measured by dynamic light scattering, (b) the particle size distribution by intensity as measured by dynamic light scattering, (c) a correlogram of the DLS data, and (d) a scanning electron micrograph image of the polymersomes.

FIG. 5 shows plots of florescence (x-axis) against frequency (y-axis) for HEK293T cells analysed by flow cytometry which had been treated with (a) polymersomes comprising PEG-PLA as the block copolymer, and a cargo of pcDNA3.1NL labelled with Cy5, (b) polymersomes comprising PEG-PDPA as the block copolymer, and a cargo of pcDNA3.1NL labelled with Cy5, and (c) a positive control comprising pcDNA3.1NL complexed to the transfection reagent JetOPTIMUS®.

FIG. 6 shows the expression levels of the NanoLuc® luciferase protein in HEK293T cells which were (1) untreated, (2) treated for 48 hours with polymersomes comprising PEG-PLA as the block copolymer, and free pcDNA3.1NL as the cargo, or (3) treated for 48 hours with polymersomes comprising PEG-PLA as the block copolymer, and a cargo comprising pcDNA3.1NL and histone H1.

FIG. 7 shows the expression levels of the NanoLuc® luciferase protein in HEK293T cells which were (1) untreated, (2) treated for 48 hours with polymersomes comprising PEG-PDPA as the block copolymer, and free pcDNA3.1NL as the cargo, or (3) treated for 48 hours with polymersomes comprising PEG-PDPA as the block copolymer, and a cargo comprising pcDNA3.1NL and histone H1.

### DETAILED DESCRIPTION OF THE INVENTION

### Polymersomes

The polymersomes of the present invention can be any polymersomes suitable for delivery of a drug cargo to a target site of action in vivo.

Polymersomes are synthetic vesicles formed from amphiphilic block copolymers. Examples of polymersomes are described in US 2010/0003336 A1, WO 2017/144849, WO 2017/158382, WO 2017/199023, WO 2017/191444, WO 2019/197834, WO 2020/144467 and WO 2020/225538, the contents of each of which are herein incorporated by reference in their entirety. Over the last twenty years they have attracted significant research attention as versatile carriers because of their colloidal stability, tuneable membrane properties and ability in encapsulating or integrating other molecules (for one representative review article, see Lee and Feijen; J Control Release, 2012, 161(2), 473-483, the contents of which are herein incorporated by reference in their entirety).

Polymersomes are typically nanoparticles or microparticles. A “nanoparticle”, as defined herein, is any particle between 1 and 100 nm in size. A “microparticle”, as defined herein, is any particle between 0.1 and 100 μm in size. The polymersomes of the present invention may be of any feasible geometry, e.g. substantially spherical, ellipsoidal, cylindrical or bilayer form, but typically they are substantially spherical. A typical (largest) diameter of a polymersome of the present invention is in the range 50 to 5000 nm. More typically, the z-average diameter, as measured using dynamic light scattering, is in the range 50 to 1000 nm. The z-average diameter is the intensity weighted mean hydrodynamic size of the particles measured (for further information on how to calculate a z-average diameter, see https://www.malvernpanalytical.com/en/learn/knowledge-center/faqs/faq0015averagediameter, the contents of which are incorporated herein by reference in their entirety). Typically, the polymersomes of the present invention have a z-average diameter of less than 300 nm, preferably less than 250 nm, most preferably less than 200 nm or 150 nm. Preferably, the polymersomes of the present invention have a z-average diameter measured by dynamic light scattering of from 50 to 150 nm, more preferably from 80 to 120 nm, e.g. approximately 100 nm. In one aspect, the polymersome of the present invention is a nanoparticle. Alternatively, the polymersome of the present invention is a microparticle. Typically, particle size is measured using transmission electron microscopy (TEM). Typically, particle size distribution is measured using dynamic light scattering (DLS).

Polymersomes are typically self-assembled structures. Polymersomes typically comprise an amphiphilic block copolymer, i.e. a block copolymer that comprises a hydrophilic block and a hydrophobic block. For example, the polymersome may comprise at least two such amphiphilic block copolymers, which are different from one another.

Such copolymers are able to mimic biological phospholipids. Molecular weights of these polymers are much higher than naturally-occurring phospholipid-based surfactants such that they can assemble into more entangled membranes (Battaglia et al.; J. Am. Chem. Soc., 2005, 127, 8757-8764, the contents of which are herein incorporated by reference in their entirety), providing a final structure with improved mechanical properties and colloidal stability. Furthermore, the flexible nature of the copolymer synthesis allows the application of different compositions and functionalities over a wide range of molecular weights and consequently of membrane thicknesses. Thus the use of these block copolymers as delivery vehicles offers significant advantages.

Polymersomes are often substantially spherical. Polymersomes typically comprise an amphiphilic membrane. The membrane is generally formed from two monolayers of amphiphilic molecules, which align and entangle to form an enclosed core with hydrophilic head groups facing the core and the exterior of the vesicle, and hydrophilic tail groups forming the interior of the membrane.

The thickness of the bilayer is generally between 2 and 100 nm, more typically between 2 and 50 nm (for instance between 5 and 20 nm). These dimensions can routinely be measured, for example by using Transmission Electron Microscopy (TEM) and/or and Small Angle X-ray Scattering (SAXS) (see, for example, Battaglia et al.; J. Am. Chem. Soc., 2005, 127, 8757-8764, the contents of which are herein incorporated by reference in their entirety).

Polymersomes of the present invention may be formed from one type of copolymer, or from more than one type of copolymer, e.g. two, three, four, five or six different types of copolymer. Typically, therefore, the polymersomes are formed from one type of copolymer. Alternatively, the polymersomes are formed from more than one type of copolymer, e.g. from two to six types of copolymer. When a polymersome is formed from more than one different type of copolymer, different regions of the polymersome typically have different bilayer thicknesses. For example, if a polymersome is formed from two different types of copolymer, preferably the thickness of the polymersome bilayer of a first region is from 1 to 10 nm, more preferably from 2 to 5 nm. Preferably the thickness of the polymersome bilayer of a second region is from 5 to 50 nm, for instance from 10 to 40 nm. More preferably the thickness of the polymersome bilayer of the second region is from 5 to 20 nm. Preferably the thickness of the polymersome bilayer of the first region is less than the thickness of the polymersome bilayer of the second region. Alternatively, the copolymers can have same thickness but different chemical compositions, which in turn create two different permeabilities with one copolymer forming a bilayer which is less permeable than the other.

In aqueous solution, normally an equilibrium exists between different types of structures, for instance between polymersomes and micelles. It is preferred that at least 80%, more preferably at least 90% or 95% by weight and most preferably all of the structures in solution are present as polymersomes. This can be achieved using the methods outlined herein.

It is known that when two different polymersome-forming copolymers are mixed to form a hybrid vesicle they phase-separate and thus give rise to polymersomes that contain discrete regions corresponding to the discrete copolymers. For example, this phenomenon is described in detail in LoPresti et al.; ACS NANO, 2011, 5(3), 1775-1784, the contents of which is herein incorporated by reference in their entirety. Polymersomes can be readily manufactured by applying these known synthetic principles.

A polymersome is preferably capable of dissociating and releasing the encapsulated cargo once it has reached the tissue of interest (i.e. the target tissue). Non-limiting, exemplary tissues of interest are discussed in more detail later and include cancer cells, immune cells and cells (e.g. CNS cells) beyond the blood-brain barrier. Preferably the polymersome is capable of dissociating and releasing the encapsulated cargo after it has been internalised, via endocytosis, within a target cell (e.g. a cancer cell, an immune cell, or a CNS cell).

Dissociation may be promoted by a variety of mechanisms, such as pH sensitivity of the block copolymer, thermal sensitivity of the block copolymer, hydrolysis (i.e. water sensitivity of the block copolymer) and/or redox sensitivity of the block copolymer.

The hydrophobic block of a copolymer comprised in the polymersome may also comprise pendant cationisable moieties as pendant groups. Cationisable moieties are, for instance, primary, secondary or tertiary amines as well as imidazole groups, capable of being protonated at pHs below a value in the range 3 to 6.9. Alternatively the group may be a phosphine.

Preferably, the degree of polymerisation of the hydrophobic block is at least 5, preferably at least 10, and more preferably at least 20. Preferably, the degree of polymerisation of the hydrophobic block is at most 500, more preferably at most 400, and most preferably at most 300. A preferred degree of polymerization of the hydrophobic block is thus 5 to 500, more preferably 10 to 400, and most preferably 20 to 300. It is preferred that the ratio of the degree of polymerisation of the hydrophilic to hydrophobic block is in the range 1:2.5 to 1:8.

The hydrophilic polymer block is not particularly limited and can, in general, be constituted from any hydrophilic polymerizable monomer such as those well-known and widely used in the art for producing amphiphilic polymers, e.g. for use in constructing micelles and/or polymersomes. A wide variety of suitable such hydrophilic polymers blocks are described in documents such as US 2010/0003336 A1, WO 2017/144849, WO 2017/158382, WO 2017/199023, WO 2017/191444, WO 2019/197834, WO 2020/144467 and WO 2020/225538, the contents of each of which are herein incorporated by reference in their entirety.

By way of example, the hydrophilic block may be based on condensation polymers, such as polyesters, polyamides, polyanhydrides, polyurethanes, polyethers (including polyalkylene glycols, especially polyethylene glycol (PEG)), polyimines, polypeptides (e.g., polysarcosine), polypeptoids, polyureas, polyacetals and polysaccharides. Preferably, the hydrophilic block is based on a polymer selected from a poly(alkylene glycol), poly(vinyl pyrrolidone) (PVP), a phosphorylcholine such as poly(2-methacryloyloxyethyl phosphorylcholine) (PMPC), poly(glycerol)s, poly(amino acid)s, polysarcosine, poly(2-oxazoline)s, poly[oligo(ethylene glycol) methyl methacrylate] and poly(N-(2-hydroxypropyl)methacrylamide). More preferably, the hydrophilic block is based on PEG, poly(propylene glycol) or poly[oligo(ethylene glycol) methyl methacrylate]. Most preferably, the hydrophilic block is based on PEG. The hydrophilic block may have zwitterionic pendant groups, in which case the zwitterionic pendant groups may be present in the monomers and remain unchanged in the polymerisation process. It is alternatively possible to derivatise a functional pendant group of a monomer to render it zwitterionic after polymerisation. The hydrophilic block may comprise one polymer or a range of different polymers.

Amphiphilic polymers of the present invention may comprise any of the structural and/or functional features of the polymersomes described in any of US 2010/0003336 A1, WO 2017/144849, WO 2017/158382, WO 2017/199023, WO 2017/191444, WO 2019/197834, WO 2020/144467 and WO 2020/225538, the contents of each of which are herein incorporated by reference in their entirety.

In one embodiment of this invention, the monomer from which the hydrophobic block is formed is 2-(diisopropylamino)ethyl methacrylate (DPA), 2-(diethylamino)ethyl methacrylate (DEA) or lactic acid (LA).

In another embodiment, the hydrophobic block is formed from 2-(diisopropylamino)ethyl methacrylate (DPA), 2-(diethylamino)ethyl methacrylate (DEA) or lactic acid (LA) and the hydrophilic block is based on a polyester, polyamide, polyanhydride, polyurethane, polyether, polyimine, polypeptide, polypeptoid, polyurea, polyacetal or polysaccharide. Preferably, the hydrophobic block is formed from 2-(diisopropylamino)ethyl methacrylate (DPA), 2-(diethylamino)ethyl methacrylate (DEA) or lactic acid (LA) and the hydrophilic block is based on PEG, poly(propylene glycol) or poly[oligo(ethylene glycol) methyl methacrylate]. More preferably, a polymersome of the present invention comprises di-block PEG-PDPA, wherein PEG is poly(ethylene glycol), and PDPA is poly(2-(diisopropylamino)ethyl methacrylate). Alternatively, a polymersome of the present invention comprises di-block PEG-PLA, where PEG is poly(ethylene glycol), and PLA is poly(lactic acid). Alternatively, a polymersome of the present invention comprises di-block POEGMA-PDPA, wherein POEGMA is poly[oligo(ethylene glycol) methyl methacrylate], and the PDPA is poly(2-(diisopropylamino)ethyl methacrylate). Most preferably, a polymersome of the present invention comprises PEG-PDPA or PEG-PLA. These copolymers have the ability to self-assemble in water or PBS and create vesicles having an aqueous lumen into which drugs can be loaded. The PEG functionality provides pendant hydroxyl groups, which act as handles for easy/reliable functionalisation of the polymers with ligands (as discussed below), while avoiding protein opsonization (giving polymersomes long circulation time and low unspecific binding). PDPA, meanwhile, is a pH-sensitive block that triggers the disassembly of polymersomes at pH values below 6.4, which is a typical pH during early stage endocytosis. PLA is also a pH-sensitive block, the degradation of which is triggered at acidic pH by hydrolysis of the ester bonds in the polymeric backbone. The pH-sensitivity allows the drug payload to be released in the cell cytosol, upon internalization of the polymersome within a cell.

The block copolymer may be a simple A-B block copolymer, or may be an A-B-A or B-A-B block linear triblock copolymer or a (A)₂B or A(B)₂ star copolymers (where A is the hydrophilic block and B is the hydrophobic block). It may also be an A-B-C, A-C-B or B-A-C block linear triblock copolymers or a ABC star copolymers (blocks linked together by the same end), where C is a different type of block. C blocks may, for instance, comprise functional, e.g. cross-linking or ionic groups, to allow for reactions of the copolymer, for instance in the novel compositions. Crosslinking reactions especially of A-C-B type copolymers, may confer useful stability on polymersomes. Cross-linking may be covalent, or sometimes, electrostatic in nature. Cross-linking may involve addition of a separate reagent to link functional groups, such as using a difunctional alkylating agent to link two amino groups. The block copolymer may alternatively be a star type molecule with hydrophilic or hydrophobic core, or may be a comb polymer having a hydrophilic backbone (block) and hydrophobic pendant blocks or vice versa. Such polymers may be formed for instance by the random copolymerisation of monounsaturated macromers and monomers.

In another embodiment, the hydrophobic polymer block is formed from fumarate. The fumarate is a typically a polymer that comprises a fumarate unit of the following formula:

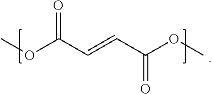

The fumarate polymer may further comprise pendant groups that are typically susceptible to degradation to release fumarate. For example, the fumarate polymer may comprise repeating units of the following formula:

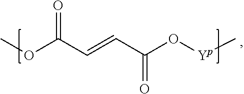

wherein Y^{p} is a straight chain or branched C₁₋₆ alkylene group. Particularly preferred repeating units are those where Y^{p} is —CH(CH₃)CH₂—. A polymer comprised of such particularly preferred repeating units is polypropylene fumarate (PPF).

Typically in these embodiments, the hydrophobic polymer block is formed from fumarate and the hydrophilic block is based on a polyester, polyamide, polyanhydride, polyurethane, polyether, polyimine, polypeptide, polypeptoid, polyurea, polyacetal or polysaccharide. Preferably in this embodiment, the hydrophobic polymer block is formed from fumarate and the hydrophilic block is formed from a phosphorylcholine polymer (e.g. PMPC). In such embodiments, the polymersome is capable of releasing fumarate in vivo after administration to a subject. Phosphorylcholine can selectively targets scavenger receptor B1 highly expressed by macrophages and other immune cells. In particular it enables the polymersome to enter such cells. In embodiments where the hydrophilic block is formed from a phosphorylcholine polymer, therefore, release of fumarate (and the encapsulated cargo within the core of the polymersome) may thus typically occur, at least in part, after the polymersome has been internalised within an immune cell. In these embodiments, dissociation of the polymersome may be promoted by a variety of mechanisms, but is often promoted by the hydrolysis of the hydrophobic block copolymer. Specifically, the ester bonds in the fumarate are hydrolysed releasing fumarate. This process is catalysed by enzymes known as esterases and by acidic pH. Both conditions are typical of endo-lysosome compartments where the polymersomes traffic to upon endocytosis. Typically, when the hydrophilic block is formed from a phosphorylcholine polymer, the phosphorylcholine polymer carries a phosphorylcholine group (typically as a pendant group), which typically has a pKa in the range 3.0 to 6.9. The process of endocytosis includes a reduction in the local pH experienced by the polymersome from around pH 7.4 to around pH 5-6. This pH drop is typically sufficient to trigger disintegration of the polymersome.

In another embodiment, the hydrophobic polymer block is formed from a pH sensitive biodegradable succinate polymer comprising a pendant group with a pKa of from 4 to 7, preferably from 5.5 to 7, and more preferably from 6.2 to 7. Preferably in this embodiment, the pendant group comprises an imidazole moiety, such as imidazole, methylimidazole or dimethylimidazole. More preferably, the pH sensitive biodegradable succinate polymer is poly[propylene 2-((2-(2-(1H-imidazol-5-yl)acetamido)-3-(tert-butoxy)-3-oxopropyl)thio)succinate]. Preferably in these embodiments, the hydrophilic block is based on polymers such as polyesters, polyamides, polyanhydrides, polyurethanes, polyethers (including polyalkylene glycols, especially PEG), polyimines, polypeptides, polyureas, polyacetals, polysaccharides and phosphorylcholine polymers (e.g. PMPC). Preferably in this embodiment, the hydrophilic block is based on phosphorylcholine polymers. These polymers are protein repellent and can selectively target receptors expressed on the cell surface membrane of target cells. Preferably in this embodiment, the hydrophobic polymer block is formed from poly[propylene 2-((2-(2-(1H-imidazol-5-yl)acetamido)-3-(tert-butoxy)-3-oxopropyl)thio)succinate] and the hydrophilic block is formed from PMPC.

In another embodiment, the hydrophobic polymer blocks comprise a polypeptide or polypeptoid. A polypeptide is a polymer block comprising or consisting of amino acid residues. A polypeptoid is a block comprising of consisting of N-substituted glycine residues.

As is very well known in the art, an amino acid is a compound that comprises at least one amine functional group and at least one carboxylic acid functional group (i.e., a group of formula —CO₂H). Usually the amino acid is an α-amino acid, although an amino acid can also, for example, be a β-amino acid or a γ-amino acid or a δ-amino acid. The amino acid may be naturally occurring, synthetic, proteogenic, or nonproteogenic.

As is well known, many amino acids have chiral centres. It is not important for the purposes of the invention whether the amino acid is chiral or achiral, or whether it is present in a particular enantiomeric form.

Examples of α-amino acids include the well-known 20 standard amino acids (specifically, Ala, Cys, Asp, Glu, Phe, Gly, His, Ile, Lys, Leu, Met, Asn, Pro, Gln, Arg, Ser, Thr, Val, Trp and Tyr) as well as other proteinogenic amino acids (e.g. fMet, Sec, Pyl) and also non-proteinogenic amino acids. The amino acid is most often an L-amino acid but may also be a D-amino acid. An amino acid may comprise a chemical modification, including but not limited to a natural post-translation modification.

In general, α-amino acid residues that can be present in the polypeptide may have the general formula —OC(═O)—CH(R)—NH— wherein R is an organic side group (substituent). There is no particular limitation on the chemical structure of the group R, which may or may not correspond to the side group of the standard and/or proteinogenic acids but may also correspond to that of known non-natural amino acids or other groups, e.g. pendant groups such as those described elsewhere herein. Those skilled in the art would readily appreciate that the invention can routinely be carried out with a wide range of amino acids and could readily implement the principles of the invention to prepare amphiphilic copolymers having polypeptide polymer blocks composed of any amino acids.

Notwithstanding the generality of the invention with respect to applicable polypeptides, non-limiting examples of preferred polypeptides are those comprising amino acid residues selected from the group consisting of methionine, histidine, lysine, glutamic acid, phenylalanine and derivatives thereof. Derivatives of these amino acid residues are not limited, but include, for instance, derivatives of lysine and glutamic acid that incorporate an imidazolyl substituent, and derivatives of glutamic acid in which the carboxylic acid side group is protected (e.g. esterified).

In one embodiment, the polypeptide comprises or consists of methionine residues. Polymersomes comprising a polymethionine segment may have particularly useful properties in the context of drug delivery applications, as they can dissociate in response to a change in concentration of reactive oxygen species (ROS) in vivo.

In another embodiment, the polypeptide comprises pendant groups having a pKa in the range 4.0 to 7.5. For instance, such pendant groups can be provided by incorporating amino acid residues that bear such pendant groups. One example of suitable such amino acids are histidine residues, as well as other amino acids functionalised to contain imidazolyl groups (lysine and glutamic acid are particularly amenable to such functionalisation). Other suitable pendant groups are those comprising primary, secondary or tertiary amines, or phosphines.

In still further embodiments, the polypeptide comprises chemically reactive pendant groups suitable for further functionalising the self-assembled nanoparticles or microparticles. Such groups may be nucleophilic or electrophilic groups. They may be used, for instance, to attach a diverse range of additional functional moieties, including but by no means limited to antibodies (and antigen-binding fragments thereof), drugs, imaging agents/labels, additional polymers, and so on.

N-substituted glycine residues can be any substance derived from glycine residues by substitution of its N-atom hydrogen substituent with an organic substituent R. Polypeptoids are known analogues of polypeptides which can provide complementary properties thereto.

In general, N-substituted glycine residues that can be present in the polypeptoid may have the general formula —OC(═O)—CH₂—NR— wherein R is an organic side group (substituent). As with the amino acid residues discussed above, there is no particularly limitation on the chemical structure of the group R. Those skilled in the art would readily appreciate that the invention can routinely be carried out with a wide range of polypeptoids and could readily implement the principles of the invention to prepare amphiphilic copolymers having polypeptoid polymer blocks composed of any N-substituted glycine residues.

Further details of a suitable process for polymerising the monomers are to be found in WO 03/074090, the contents of which are herein incorporated by reference in their entirety.

Exemplary methods that can be used for polymerising the monomers are atom-transfer radical polymerisation (ATRP) (see, e.g., an exemplary method described in Journal of the American Chemical Society 127, 17982-17983), living radical polymerisation process, functional NCA (N-carboxyanhydride) polymerisation with efficient postpolymerization modification and ring opening polymerisation (ROP). Living radical polymerisation has been found to provide polymers of monomers having a polydispersity (of molecular weight) of less than 1.5, as judged by gel permeation chromatography. Polydispersities in the range of from 1.2 to 1.4 for the or each block are preferred. The polymersomes may be loaded using a pH change system, electroporation or film hydration. In a pH change system process, polymer is dispersed in aqueous liquid in ionised form, in which it solubilises at relatively high concentrations without forming polymersomes. Subsequently the pH is changed such that some or all of the ionised groups become deprotonated so that they are in non-ionic form. At the second pH, the hydrophobicity of the block increases and polymersomes are formed spontaneously.

A method of forming polymersomes with the encapsulated cargo may involve the following steps: (i) dispersing the amphiphilic copolymer in an aqueous medium; (ii) acidifying the composition formed in step (i); (iii) adding the cargo to be encapsulated to the acidified composition; and (iv) raising the pH to around neutral to encapsulate the cargo.

This method preferably comprises a preliminary step wherein the amphiphilic copolymer is dispersed in an organic solvent in a reaction vessel and the solvent is then evaporated to form a film on the inside of the reaction vessel.

Step (ii), of acidifying the composition, typically reduces the pH to a value below the pKa of the pendant group.

Another method of forming polymersomes with an encapsulated cargo in the core may involve the following steps: (i) dispersing the amphiphilic copolymer, and when needed the material to be encapsulated, in an organic solvent (e.g. a 2:1 chloroform:methanol mixture) in a reaction vessel; (ii) evaporating the solvent to form a film on the inside of the reaction vessel; and (iii) re-hydrating the film with an aqueous solution, optionally comprising a solubilised material to be encapsulated.

Another method of forming polymersomes with an encapsulated material in the core may involve the following steps: (i) dispersing the amphiphilic copolymer, and when needed the material to be encapsulated, in an organic solvent in a reaction vessel; (ii) adding an aqueous solvent to enable solvent switch and the formation of polymersomes on the inside of the reaction vessel; and (iii) optionally electroporating the obtained polymersomes to allow encapsulation of water-soluble bioactive molecules.

Another method of forming polymersomes with an encapsulated material in the core may involve the following steps: (i) dispersing the amphiphilic copolymer, and when needed the material to be encapsulated, in an organic solvent in a reaction vessel; (ii) adding a different organic solvent to enable solvent displacement and the formation of polymersomes on the inside of the reaction vessel; and (iii) optionally electroporating the obtained polymersomes to allow encapsulation of water-soluble bioactive molecules.

Another method of forming polymersomes with an encapsulated material in the core may involve the following steps: (i) dispersing the amphiphilic copolymer, and when needed the material to be encapsulated, in an organic solvent in a reaction vessel; (ii) adding this solution to an aqueous solvent to enable solvent displacement and the formation of polymersomes on the inside of the reaction vessel; and (iii) optionally electroporating the obtained polymersomes to allow encapsulation of water-soluble bioactive molecules.

UV spectroscopy and HPLC chromatography may be used to calculate the encapsulation efficiency of a polymersome, using techniques well known in the art. An alternative method for forming polymersomes with an encapsulated material may involve simple electroporation of the material and polymer vesicles in water. For instance the drug may be contacted in solid form with an aqueous dispersion of polymer vesicles and an electric field applied to allow the formation of pores on the polymersomes membrane. The solubilised material molecules may then enter the polymersome vesicles though the pores. This is followed by membrane self-healing process with the consecutive entrapment of the material molecules inside the polymersomes.

Alternatively, material dissolved in organic solvent may be emulsified into an aqueous dispersion of polymer vesicles, whereby solvent and the material become incorporated into the core of the vesicles, followed by evaporation of solvent from the system. The polymersomes of the present invention may be formed from two or more different block copolymers. In this embodiment, in the method of forming polymersomes, a mixture of the two or more block copolymers is used.

For example, 0.01% to 10% (w/w) of material to be encapsulated is mixed with copolymer in the methods described above.

In the case where the hydrophobic block comprises a polypeptide or polypeptoid, a preferred method of preparing the polymersomes comprises:
(i) providing, in a polar aprotic solvent, hydrophilic polymer blocks as initiator molecules;
(ii) contacting said hydrophilic polymer blocks with hydrophobic polymer block precursor monomers, and forming said hydrophobic polymer blocks by polymerization reactions, initiated at the hydrophilic polymer blocks, of said hydrophobic polymer block precursor monomers, thereby producing amphiphilic copolymers; and
(iii) allowing said amphiphilic copolymers to self-assemble in situ to form said polymersomes.

Preferably this method further comprises transferring (e.g., by membrane dialysis, ultrafiltration, size exclusion chromatography or tangential flow filtration) said polymersomes into an aqueous medium by displacement of the polar aprotic solvent.

In an embodiment of this method, the hydrophobic polymer block precursor monomers are typically cyclic and the polymerization reactions are ring-opening polymerization (ROP) reactions. For instance, the hydrophobic polymer block precursor monomers may be amino acid N-carboxyanhydrides.

In an embodiment of this method, the hydrophobic polymer block comprises a polypeptide. For instance, in exemplary aspects the polypeptide comprises amino acid residues selected from the group consisting of methionine, histidine, lysine, glutamic acid, phenylalanine and derivatives thereof. Particularly preferred polypeptides include: (a) a polypeptide that comprises methionine; (b) a polypeptide that comprises pendant groups having a pKa in the range 4.0 to 7.5; and/or (c) a peptide that comprises chemically reactive pendant groups suitable for further functionalising the self-assembled nanoparticles or microparticles.

In an embodiment of this method, the hydrophilic polymer block comprises a polymer selected from the group consisting of polyesters, polyamides, polyanhydrides, polyurethanes, polyethers, polyimines, polypeptides, polypeptoids, polyureas, polyacetals and polysaccharides.

In an embodiment of this method, the polar aprotic solvent is selected from the group consisting of dimethyl sulfoxide, tetrahydrofuran, dioxane, N,N-dimethyl formamide, N,N-dimethyl acetamide and 1,3-dimethyl-2-imidazolidinone.

Optionally, the method comprises providing said hydrophilic polymer blocks in situ by polymerizing hydrophilic polymer block precursor monomers.

In a particularly preferred embodiment of this method, the steps (i) to (iii) are carried out as a one-pot reaction.

In an embodiment, when the hydrophobic block is a polypeptide or polypeptoid, polymerization of the monomer units to form the hydrophobic block can be effected by ring-opening polymerization reactions using amino acid N-carboxyanhydrides. In the context of the non-limiting α-amino acid and N-substituted glycine residues described above, such cyclic N-carboxyanhydride (NCA) reagents may have the structure

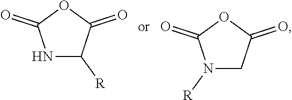

respectively.

In one embodiment of the method for preparing such polymersomes, step (ii) comprises providing the NCAs as reagents, i.e. a physical step of providing the NCAs and mixing them with the hydrophilic polymer blocks (or the hydrophilic polymer block precursor monomers where the hydrophilic polymer blocks are to be formed in situ).

Alternatively, as is known in the art, NCAs are capable of forming in situ from suitably activated amino acids (or N-substituted glycines). Thus, step (ii) of the method may comprise allowing the hydrophilic polymer blocks to contact hydrophobic polymer block precursor monomers (specifically NCAs or N-substituted glycines) formed in situ from activated amino acid molecules. In this case, the method may comprise a physical step of providing the activated amino acid (or N-substituted glycines) molecules and mixing them with the hydrophilic polymer blocks (or the hydrophilic polymer block precursor monomers where the hydrophilic polymer blocks are to be formed in situ).

There is no particular limitation on the structure of such activated amino acids or N-substituted glycines, provided that they are capable of forming corresponding NCAs in situ. However, in a preferred method the activated molecules are urethane derivatives of the corresponding amino acid or N-substituted glycine, i.e. in which a hydrogen attached to the (non-side chain) amino group of the relevant amino acid is replaced by an activating group that transforms the amino group into a urethane group (i.e. —C(O)—O—R′ replaces the —H that is attached to the amino group in the relevant amino acid; R′ can be any suitable substituent, including but not limited to halogen atoms and organic and hydrocarbyl groups). Such urethane derivatives of amino acids and N-substituted glycines are known in the art to be susceptible to cyclization to form NCAs under mild conditions (see, e.g., Doriti, Polym. Chem., 2016,7, 3067-3070).

In an embodiment, the polymersomes of the present invention comprise a hydrophobic block that is capable of undergoing a chemical transformation, leading to degradation of the polymersome in vivo, in response to a change in in vivo conditions. The ability of the polymersome to degrade in this way may be of significant utility in drug delivery applications. The relevant change in in vivo conditions can be any of a range of phenomena, including a change in pH (for instance, associated with the process of endocytosis), a change in concentration of reactive oxygen species, ROS (for instance, high concentrations of ROS may be present at a disease site), or a change in temperature.

In one such embodiment, the change in in vivo conditions is an increase in the concentration of reactive oxygen species (ROS). In this embodiment, an exemplary component of the hydrophobic block is methionine residues. In another embodiment, the change in in vivo conditions is a change in pH and the hydrophobic block comprises pendant groups having a pKa in the range 4.0 to 7.5. Examples of hydrophobic blocks comprising such pendant groups are described elsewhere herein. In one embodiment, for instance, the pendant groups may comprise imidazolyl groups and preferably at least some of these imidazolyl groups are provided by histidine residues in the hydrophobic block.

### Alternative Nanoparticles and Microparticles

In some embodiments, the microparticle or nanoparticle is other than a polymersome. In such embodiments, the microparticle or nanoparticle may be any object able to encapsulate a nucleic acid and a nucleic acid-binding protein. For example, the microparticle or nanoparticle may be a liposome, a synthosome, latex, a micelle, a nanocrystal, a quantum dot, a metallic nanoparticle, an oxide nanoparticle, a silica nanoparticle, a protein cage, a nano- or micro-gel, a dendrimer, or a virus-like particle. These alternative nanoparticles and microparticles typically have the same preferred shapes, dimensions etc. as described above in respect of the polymersomes of the present invention.

A nanoparticle or microparticle may be a liposome. A liposome is a spherical vesicle having at least one lipid bilayer. Typically, a liposome comprises a phospholipid, e.g. phosphatidylcholine, but may also include other lipids, such as egg phosphatidylethanolamine, so long as they are compatible with a lipid bilayer structure. The major types of liposomes include the multilamellar vesicle (MLV, with several lamellar phase lipid bilayers), the small unilamellar liposome vesicle (SUV, with one lipid bilayer), the large unilamellar vesicle (LUV), and the cochleate vesicle.

Typically, liposomes are fusogenic liposomes. This means that they are capable of fusing with a membrane, e.g. the cell surface membrane of a target cell, or the membrane of an endosome within the cell. Fusion of the bilayer of a fusogenic liposome with the cell surface membrane results in the incorporation of the liposome bilayer into the cell surface membrane, and the release of the drug cargo contained within the lysosome into the cell cytosol. Alternatively, the liposome may be internalized within a target cell via endocytosis, and the drug cargo carried within the liposome is released after fusion of the liposome bilayer with the endosomal membrane. The pH within an endosome is slightly acidic and therefore it is advantageous for the liposomes to be pH sensitive, e.g. the stability of the liposome structure is decreased at lower pH, facilitating fusion with the endosomal membrane. Other environments having low pH can also trigger the fusion of such liposomes, e.g., the low pH found in tumors or sites of inflammation.

Liposomes may be zwitterionic structures. Alternatively, liposomes may be amphoteric liposomes. This means that the liposomes have an isoelectric point and are negatively charged at higher pH values and positively charged at lower pH values. Typical pH-responsive elements in pH-sensitive liposomes include cholesterol hemisuccinate (CHEMS), palmitoylhomocysteine, dioleoylglycerol hemisuccinate (DOG-Succ) and the like.

Alternatively, the nanoparticle or microparticle may be a synthosome. Synthosomes are a particular type of polymersome engineered to contain channels (transmembrane proteins) that selectively allow certain chemicals to pass through the membrane, into or out of the vesicle.

Alternatively, the nanoparticle or microparticle may be a micelle. Micelles are aggregates (or supramolecular assemblies) of molecules having both hydrophilic and hydrophobic regions, dispersed in a liquid. Typically in an aqueous solution, the aggregated micelle is arranged such that the hydrophobic regions of the molecules are sequestered in the centre of the micelle, whilst the hydrophilic regions of the molecules present on the external surface of the micelle, and contact the aqueous solvent. Typically, micelles are substantially spherical in shape, although other shapes such as ellipsoid, cylindrical, torus and discoid are also possible.

Alternatively, the nanoparticle or microparticle may be any object able to encapsulate and/or conjugate any type of bioactive molecules, such as anticancer drugs, proteins, peptides (natural or not), antibodies, fragment of antibodies, dyes, and the like.

### Nucleic Acid Cargoes

The polymersomes of the present invention comprise a cargo which comprises a nucleic acid and a nucleic acid-binding protein. Typically, the cargo is a complex of a nucleic acid and a nucleic acid-binding protein.

The nucleic acid is typically selected from a ribonucleic acid (an RNA), a deoxyribonucleic acid (a DNA) or a peptide nucleic acid (a PNA). Preferably, the nucleic acid is selected from an RNA and a DNA. RNAs may be single-stranded or double-stranded RNAs and include messenger RNA (mRNA), short interfering RNA (siRNA), short hairpin RNA (shRNA), microRNA (miRNA), transfer RNA (tRNA) and RNA-based antisense oligonucleotides (ASOs). DNAs may be single-stranded or double-stranded DNAs and include plasmid DNA (pDNA) and DNA-based antisense oligonucleotides (ASOs).

Plasmid DNA cargo typically comprises from 2,000 to 400,000 base pairs, more preferably from 2,000 to 12,000 base pairs, and most preferably from 2,000 to 6,000 base pairs. Double-stranded DNA cargoes typically comprise from 100 to 5000 base pairs.

Typically, single-stranded messenger RNA cargo comprises from 300 to 3000 nucleotides, depending on the identity of the delivered gene. A single strand of other nucleic acid cargoes typically comprises from 3 to 300 nucleotides, preferably from 5 to 100 nucleotides, more preferably from 10 to 50 nucleotides, still more preferably from 15 to 30 nucleotides, and most preferably from 20 to 25 nucleotides.

As used herein, the term “DNA” refers to deoxyribonucleic acid and derivatives thereof, the molecule that carries most of the genetic instructions used in the development, functioning and reproduction of all known living organisms and many viruses. Most DNA molecules consist of two biopolymer strands coiled around each other to form a double helix. The two DNA strands are known as polynucleotides since they are composed of simpler units called nucleotides. Each nucleotide is composed of a nitrogen-containing nucleobase—cytosine (C), guanine (G), adenine (A), or thymine (T)—as well as a monosaccharide sugar called deoxyribose and a phosphate group. The nucleotides are joined to one another in a chain by covalent bonds between the sugar of one nucleotide and the phosphate of the next, resulting in an alternating sugar-phosphate backbone. According to base pairing rules (A with T, and C with G), hydrogen bonds bind the nitrogenous bases of the two separate polynucleotide strands to make double-stranded DNA.

As used herein, the term “plasmid DNA” (pDNA) refers to a small, extrachromosomal DNA molecule that is physically separated from chromosomal DNA and can replicate independently. Typically, a plasmid is a small, circular, double-stranded helix of DNA. Typically, a plasmid encodes at least one (or more than one, e.g. two, three, four, five, six or more) gene. Plasmid DNA may be used in medical applications to induce translation and transcription of a gene within a target cell to which the plasmid is introduced. This can therefore result in an increase in the levels of a particular protein, for which the plasmid DNA codes, within the cell. For example, plasmids may be used for gene transfer as a potential treatment in gene therapy, so that the target cell may express a protein that is lacking in the cells. A plasmid may also be referred to as a “vector”.

As used herein, the term “RNA” refers to ribonucleic acid and derivatives thereof. RNA molecules are essential in various biological roles in coding, decoding, regulation and expression of genes. Unlike DNA, RNA is typically a single-stranded molecule folded onto itself, although in some cases RNA may be paired to form a double strand (e.g. in siRNA). Each RNA nucleotide is composed of a nitrogen-containing nucleobase—cytosine (C), guanine (G), adenine (A), or uracil (U)—as well as a monosaccharide sugar called deoxyribose and a phosphate group. In some cases, RNA nucleotides may contain a modified nucleobase, such as pseudouracil or N6-methyladenine. The nucleotides are joined to one another in a chain by covalent bonds between the sugar of one nucleotide and the phosphate of the next, resulting in an alternating sugar-phosphate backbone.

As used herein, the term “mRNA” refers to messenger RNA, a family of RNA molecules that convey genetic information from DNA to the ribosome, where they specify the amino acid sequence of the protein products of gene expression. Following transcription of primary transcript mRNA (known as pre-mRNA) by RNA polymerase, processed, mature mRNA is translated into a polymer of amino acids: a protein. As in DNA, mRNA genetic information is in the sequence of nucleotides, which are arranged into codons consisting of three bases each. Each codon encodes for a specific amino acid, except the stop codons, which terminate protein synthesis. This process of translation of codons into amino acids requires two other types of RNA: transfer RNA (tRNA), that mediates recognition of the codon and provides the corresponding amino acid, and ribosomal RNA (rRNA), that is the central component of the ribosome's protein-manufacturing machinery.

As used herein, the term “small interfering RNA” (siRNA) refers to a class of double-stranded RNA molecules, which are typically 20-25 base pairs in length. siRNA plays many roles, but it is most notable in the RNA interference (RNAi) pathway, where it interferes with the expression of specific genes with complementary nucleotide sequences. siRNA functions by causing mRNA to be broken down after transcription, resulting in no translation. siRNA also acts in RNAi-related pathways, e.g. as an antiviral mechanism or in shaping the chromatin structure of a genome.

As used herein, the term “small hairpin RNA” (shRNA) refers to an artificial RNA molecule with a tight hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover.

As used herein, the term “micro RNA” (miRNA) refers to a small non-coding RNA molecule (containing about 22 nucleotides) found in plants, animals, and some viruses, which functions in RNA silencing and post-transcriptional regulation of gene expression. As used herein, the term “antisense oligonucleotide” (ASO) refers to a single strand of DNA or RNA that is complementary to a chosen sequence. Such oligonucleotides prevent translation of certain messenger RNA strands by hybridizing to them using standard Watson-Crick base pairing rules. Antisense oligonucleotides can be used to target a either a coding or non-coding RNA. This mechanism of action is therefore distinct from nucleic acids which act via RNA interference (e.g. siRNAs, miRNAs and shRNAs). Hybrids comprising ASOs can be degraded by the enzyme RNase H.

As used herein, the term “PNA” refers to peptide nucleic acid, an artificially synthesized polymer similar to DNA or RNA invented by Peter E. Nielsen (Univ. Copenhagen), Michael Egholm (Univ. Copenhagen), Rolf H. Berg (Risø National Lab), and Ole Buchardt (Univ. Copenhagen) in 1991. PNA's backbone is composed of repeating N-(2-aminoethyl)-glycine units linked by peptide bonds. The various purine and pyrimidine bases are linked to the backbone by a methylene bridge (—CH₂—) and a carbonyl group ((C═O)—).

The nucleic acid-binding protein is selected as a suitable binding partner for the nucleic acid cargo. Proteins that are specialised to bind to each different type of nucleic acid cargo are well-known in the art and an appropriate binding protein for a specific cargo can be readily selected by the skilled person.

For instance, if the nucleic acid is a DNA, then the nucleic acid-binding protein is preferably a histone protein. Typically, in this embodiment, the nucleic acid-binding protein is a histone selected from an H1, H2A, H2B, H3 or H4 histone. H1 histones are sometimes referred to as H5 histones. Histones H2A, H2B, H3 and H4 are sometimes known as core histones, and H1/H5 histones are known as linker histones. Typically, therefore, if the nucleic acid is a DNA, the nucleic acid-binding protein may be a linker histone. Alternatively, the nucleic acid-binding protein may be a core histone. Preferably, the histone is selected from H1 and H3. Most preferably, the histone is H1. In a particularly preferred embodiment, the nucleic acid cargo is a plasmid DNA (pDNA) and the nucleic acid-binding protein is a histone, preferably H1 or H3, and more preferably H1. Alternatively, the nucleic acid cargo is a DNA-based antisense oligonucleotide and the nucleic acid-binding protein is a histone, preferably H1 or H3, and more preferably H1. Alternatively, the nucleic acid cargo is a single-stranded DNA (ssDNA) and the nucleic acid-binding protein is a histone, preferably H1 or H3, and more preferably H1.

If the nucleic acid is an RNA, then the nucleic acid-binding protein is preferably a eukaryotic translation initiation factor 4E (EIF4E) or a polyA binding protein. In one embodiment, the nucleic acid cargo is an mRNA, and the nucleic acid-binding protein is EIF4E or a polyA binding protein.

### Targeting Ligands

In certain embodiments, the cargo-containing polymersome of the present invention is for binding to the surface of a cell and comprises (a) a polymer brush and (b) at least a first ligand type on its external surface, wherein said first ligand type is capable of binding to a first receptor type on said cell surface. This aids the binding of the polymersomes to a target cell, thus improving binding to, and internalisation within, the target cell.

A “ligand” may also be referred to herein as a “targeting moiety”. By “on its external surface” is meant that each ligand is located such that it is able to interact with its target (as opposed to being located at an inaccessible position that precludes interaction with the target, for example by being encapsulated within the nanoparticle or microparticle).

In further embodiments, the cargo-containing polymersome of the present invention may additionally comprises at least a second ligand type on its external surface, wherein said second ligand type is capable of binding to a second receptor type on said cell surface. Without wishing to be bound by any particular theory, it is believed that the multiplexing of ligands on the surface of a nanoparticle or microparticle in this fashion confers the property of “super-selectivity” for the target cells. In other words, such “multiplexed” polymersomes provide the advantage that polymersome-cell binding via multiple different ligand types results in enhanced selectivity in delivery of the encapsulated cargo carried within the polymersome, reducing undesired off-target binding. A further advantage of this embodiment is that by using a multiplexed polymersome, the cell surface receptors on a target cell (e.g. a cancer cell or an immune cell) are less readily able to mutate in such a fashion that would prevent effective binding of the polymersome over time. The concept of “super-selectivity” of polymersomes is discussed in detail in WO 2020/144467, the contents of which are incorporated herein by reference in their entirety.

In some embodiments, therefore, the polymersome of the present invention is for binding to the surface of a cell, and comprises at least a first ligand type on its external surface and at least a second ligand type on its external surface, wherein said first ligand type is capable of binding to a first receptor type on said cell surface, and said second ligand type is capable of binding to a second receptor type on said cell surface. The polymersome of the present invention may, for example, comprise from two to seven different ligand types on its external surface (e.g. from two to six different ligand types, or from three to five different ligand types, or two ligand types), each of which is capable of binding to a complementary receptor type on the cell surface.

For super-selective interactions to be observed in the context of polymersomes, it is also advantageous that each ligand individually has a very low binding affinity for its target receptor. In practice, selective ligands with such a low binding energy to a target receptor are not readily available. This problem may, however, be overcome by also providing on the surface of the polymersomes a moiety which creates an interference steric potential with the surface of the target cell, such as a polymer brush. Typically a polymer brush may comprise a naturally occurring polymer, such as a polypeptide or polysaccharide, or a synthetic polymer, such as any of the amphiphilic block copolymers described above. Components on the external surface of the target cell, such as glycans, glycoproteins and glycolipids (collectively referred to as the “glycocalyx”), are also believed to contribute to this repulsive steric potential. Preferred polymeric components of the polymer brush include poly(ethylene glycol) (PEG), poly(vinyl pyrrolidone) (PVP), poly(2-methacryloyloxyethyl phosphorylcholine) (PMPC), poly(glycerol)s, poly(sulfobetaine), poly(carboxybetaine), poly(amino acid)s, polysarcosine, poly(2-oxazoline)s, poly(N-(2-hydroxypropyl)methacrylamide), polyglycols, heparin, dextran, poly(ethylene glycol)-poly(2-(diisopropylamino)ethyl methacrylate) and/or poly(oligo(ethylene glycol) methyl ether methacrylate) (POEGMA).

Preferably in such embodiments, the polymer brush has a degree of polymerisation of at least 5, more preferably at least 10. Preferably, the degree of polymerisation of the polymer brush is no more than 500, e.g. no more than 300, or no more than 200. Preferably, the polymer brush has a length of from 1.5 to 350 nm, and more preferably from 3 to 210 nm.

The number of each type of ligand on the external surface of the polymersome may be optimised once certain physical parameters of a ligand-receptor system (which can readily be determined by a person of skill in the art) are known. Specifically, in certain embodiments, the optimum number of ligands of the ith ligand type (li) on the external surface of the polymersome may be defined according to the following equation (1):

$$\frac{ln ( {a}^{- 1} - 1 )}{ln ( {r}_{i} {e}^{{E}_{B} ( i )} + 1 )} + \frac{30 {k}_{b} T}{{E}_{B} ( i )} \geq {l}_{i} \geq \frac{ln ( {a}^{- 1} - 1 )}{ln ( {r}_{i} {e}^{{E}_{B} ( i )} + 1 )} - \frac{30 {k}_{b} T}{{E}_{B} ( i )} \qquad ( 1 )$$

wherein:
a is the nanoparticle or microparticle activity and is calculated as a=[P]V_{P}N_{A}, wherein [P] is the molar concentration of nanoparticles or microparticles in bulk solution, N_{A} is the Avogadro constant, and V_{P} is the geometrical volume;
k_{B} is the Boltzmann constant;
T is the absolute temperature;
E_{B}(i) is the total energy of binding of the i^{th} ligand-receptor pair, given by the sum of (a) the ligand/receptor binding affinity kBTlnK_{D}(i), wherein, K_{D}(i) is the dissociation constant for the ith receptor/ligand couple, and (b) the steric interference, Eₛ, between the polymersome and the cell surface; and
rᵢ is the density of receptors of type i on the cell surface.

In the case of a spherical (or substantially spherical) particle, the parameter a can be calculated as a=[P]N_{A}(π/3)[3(R+d)³−2R³)], wherein R is the radius of the nanoparticle or microparticle, and d is the ligand tether length.

The number (and density) of each type of ligand on the external surface of a polymersome can typically be controlled during synthesis of the polymersome by varying the ratio of ligand-bound copolymer and “pristine” copolymer (i.e. diblock copolymer that does not have a ligand attached). For any given system, the number of ligands per polymersome is then given by the copolymer self-assembly parameter (related to the polymer molecular weight and the packing factor) and the polymersome size. The number of each type of ligand on the external surface of a polymersome (and hence the density of receptors) can typically be verified using mass spectrometry.

Typically in such embodiments, the ligand is attached to a polymer component on the external surface of the polymersome, i.e. the hydrophilic block of the amphiphilic diblock copolymer. Thus, the ligand tether length d is given by the molecular mass of the hydrophilic block. Typically, d=0.3N nm, where N is the polymerisation degree of the hydrophilic block.

The overall steric potential Eₛ is the sum of the steric potential arising from the glycocalyx brush on the cell surface and the steric potential arising from the polymer brush that coats the polymersome. The magnitude of both depends on how accessible the ligands and receptor are. This in turn depends on: (i) the relative height of the receptor with respect to the glycan/glycoprotein/glycolipid etc. chains on the cell surface (δ_{G}h_{G} where h_{G} is the glycan/glycoprotein/glycolipid length and δ_{G} is between 0 and 1 and is a measure of how buried the receptor is in the glycocalyx), and (ii) the tether length of the ligands relative to the length of the polymer chains of the brush on the external surface of the nanoparticle (δ_{P}h_{P} where h_{P} is the polymer chain length and δ_{P} is between 0 and 1 and is a measure of how buried the ligand is in the polymer brush). These parameters can readily be obtained for any given system from structural biology databases known in the art.

Typically in such embodiments, the polymersome comprises from 2 to 1000 ligands of the first ligand type. Preferably, the polymersome comprises from 5 to 1000 ligands of the first ligand type, more preferably from 10 to 500 ligands of the first ligand type, even more preferably from 20 to 200 ligands of the first ligand type, and most preferably from 50 to 100 ligands of the first ligand type.

Typically in such embodiments, the polymersome comprises from 2 to 1000 ligands of the second ligand type. Preferably, the polymersome comprises from 5 to 1000 ligands of the second ligand type, more preferably from 10 to 500 ligands of the second ligand type, even more preferably from 20 to 200 ligands of the second ligand type, and most preferably from 50 to 100 ligands of the second ligand type.

Typically in such embodiments, the polymersome comprises from 2 to 1000 ligands of a subsequent (i.e. third or greater) ligand type. Preferably, the polyersome comprises from 5 to 1000 ligands of the subsequent ligand type, more preferably from 10 to 500 ligands of the subsequent ligand type, even more preferably from 20 to 200 ligands of the subsequent ligand type, and most preferably from 50 to 100 ligands of the subsequent ligand type.

Typically in such embodiments, the combination of ligands on the surface of the nanoparticle or microparticle leads to a total binding energy of from 8k_{B}T to 30k_{B}T, where k_{B} is Boltzmann's constant and T is the temperature. This leads to on-off association profiles of the polymersomes wherein the receptors are saturated only above a given onset receptor density, whilst the polymersomes do not bind at all at lower receptor densities.

Typically in such embodiments, each ligand type is adapted to enable the polymersomes to bind to a target. Typically the ligand binds selectively to the target. The target is a chemical substance that is located on or in the vicinity of the tissue of interest (and thus enables the polymersome to accumulate specifically at the tissue of interest in preference to other sites). The target is preferably a receptor, e.g. a receptor that is present in particularly high quantity at the target tissue of interest. Most preferably, the target is a receptor on or within a cell surface membrane.

Typically in such embodiments, each ligand type can be any ligand that binds specifically to the target. As is well known in the art, for example from the well-developed field of bioconjugates, a wide range of substances can be used as ligands, e.g. to target receptors.

In one such embodiment, each ligand is a moiety that is attached to the external surface of the polymersomes. Examples of suitable ligands include antibodies, antibody fragments, aptamers, oligonucleotides, small molecules, peptides and carbohydrates. Peptide, protein, antibody and antibody fragment ligands are particularly preferred. However, any such moiety can be used as a ligand in the present invention. The suitability of any given moiety to target any given receptor can be determined using routine assay methods, involving testing for the ability of the moiety to bind specifically to the receptor.

One example of a ligand is a ligand that is adapted to enable the nanoparticle or microparticle to cross the blood-brain barrier (BBB). This property of the ligand arises through the ability of the ligand to bind to a target (e.g. a receptor) at the blood-brain barrier, wherein the target (e.g. receptor) mediates transcytosis across the blood-brain barrier. Examples of receptors for receptor-mediated transcytosis that are highly expressed on the endothelial cells that form the blood-brain barrier include low-density lipoprotein receptor-related protein 1 (LRP-1), scavenger receptor class B, member 1 (SCARB1), insulin receptor (IR) and transferrin receptor 1 (TFRC), all of which are suitable targets for the targeting moiety.

Thus, in one embodiment, at least one of the ligand types, and preferably one ligand type, targets the LRP-1 receptor. LRP-1 is a member of the LDL receptor family that plays diverse roles in various biological processes including lipoprotein metabolism, degradation of proteases, activation of lysosomal enzymes and cellular entry of bacterial toxins and viruses. Deletion of the LRP-1 gene leads to lethality in mice, revealing a critical, but as of yet, undefined role in development. Tissue-specific gene deletion studies reveal an important contribution of LRP-1 in the vasculature, central nervous system, in macrophages and in adipocytes. Three important properties of LRP-1 dictate its diverse role in physiology: first, its ability to recognise more than thirty distinct ligands; second, its ability to bind a large number of cytoplasmic adaptor proteins via determinants located on its cytoplasmic domain in a phosphorylation-specific manner; and third, its ability to associate with and modulate the activity of other transmembrane receptors such as integrins and receptor tyrosine kinases.

Polymersomes that feature a ligand that targets the LRP-1 receptor may cross the BBB and deliver efficiently the encapsulated cargo into both the CNS parenchyma and CNS cells. In particular, it has been found that the endothelial transcytosis mechanism does not involve acidification of the polymersome in membrane-trafficking organelles, which is important to avoid premature disintegration of the polymersome and concomitant release of the encapsulated cargo. Still further, the LRP-1 receptor is associated with traditional endocytosis in CNS cells, which, subsequent to navigation across the BBB, aids the delivery of the drug within their cytosol (via disintegration of the nanoparticle or microparticle).

Peptides that bind to the receptor LRP-1 are known in the art. For example, Angiochem (Montreal, Canada) have developed peptides that the leverage the LRP-1 mediated pathway to cross the blood-brain barrier when conjugated to drug cargos. One specific example of a peptide that is suitable for use in the present invention is Angiopep-2, which is a peptide having the sequence TFFYGGSRGKRNNFKTEEY. Further examples of suitable targeting moieties are disclosed in WO 2013/078562, the contents of which are herein incorporated by reference in their entirety (and, specifically, the ligand peptides disclosed in which are herein incorporated by reference).

In one embodiment, at least one of the ligand types, and preferably one ligand type, targets the SCARB1 receptor. The protein encoded by this gene is a plasma membrane receptor for high density lipoprotein cholesterol (HDL). The encoded protein mediates cholesterol transfer to and from HDL. In addition, this protein is a receptor for hepatitis C virus glycoprotein E2.

Malignant tumours display remarkable heterogeneity to the extent that even at the same tissue site different types of cells with varying genetic background may be found. In contrast, a relatively consistent marker the scavenger receptor type B1 (SR-B1) has been found to be consistently overexpressed by most tumour cells. Scavenger Receptor Class B Type I (SR-BI) is a high-density lipoprotein (HDL) receptor that facilitates the uptake of cholesterol esters from circulating lipoproteins. Additional findings suggest a critical role for SR-BI in cholesterol metabolism, signalling, motility, and proliferation of cancer cells and thus a potential major impact in carcinogenesis and metastasis. Recent findings indicate that the level of SR-BI expression correlate with aggressiveness and poor survival in breast and prostate cancer. Moreover, genomic data show that depending on the type of cancer, high or low SR-BI expression may promote poor survival. SR-BI is considered a diagnostic as well as prognostic indicator of cancer to help elucidate the contributions of this protein to cancer development, progression, and survival.

Ligands that bind to SCARB1 are known in the art. One such ligand is poly(2-(methacryloyloxy)ethyl phosphorylcholine) (PMPC).

In certain embodiments, one ligand type on the external surface of the polymersome targets LRP-1 and another ligand type on the external surface of the polymersome targets SCARB1.

In another embodiment, at least one of the ligand types, preferably one ligand type, is a ligand that is adapted to enable the polymersome to bind to a cancer cell. Cancer cells typically have a high density of membrane receptors. Illustrative and non-limiting examples of such targeting moieties include proteins (mainly antibodies and their fragments), peptides, nucleic acids (aptamers), small molecules, vitamins and carbohydrates.

Examples of receptors for receptor-mediated transcytosis that are highly expressed on tumour cells include LRP-1, SCARB1, TFRC, folate receptor 1 (FOLR1) and epidermal growth factor receptor (EGFR). For example, SCARB1 is highly expressed in HeLa cells (cervical cancer) and FaDu cells (squamous cell carcinoma of the hypopharynx).

In one embodiment, at least one of the ligand types, and preferably one ligand type, targets LRP-1. In another embodiment, at least one of the ligand types, and preferably one ligand type, targets SCARB1. In another embodiment, at least one of the ligand types, and preferably one ligand type, targets TFRC. In another embodiment, at least one of the ligand types, and preferably one ligand type, targets FOLR1. In another embodiment, at least one of the ligand types, and preferably one ligand type, targets EGFR.

In one embodiment, at least one of the ligand types, and preferably one ligand type, targets the TFRC receptor. This gene encodes a cell surface receptor necessary for cellular iron uptake by the process of receptor-mediated endocytosis. This receptor is required for erythropoiesis and neurologic development.

Iron as an important element plays crucial roles in various physiological and pathological processes. Iron metabolism behaves in systemic and cellular two levels that usually are in balance conditions. The disorders of the iron metabolism balances relate with many kinds of diseases including Alzheimer's disease, osteoporosis and various cancers. In systemic iron metabolism that is regulated by hepcidin-ferroportin axis, plasma iron is bound with transferrin (TF) which has two high-affinity binding sites for ferric iron. The generic cellular iron metabolism consists of iron intake, utilization and efflux. During the iron intake process in generic cells, transferrin receptors (TFRs) act as the most important receptor mediated controls. TFR1 and TFR2 are two subtypes of TFRs those bind with iron-transferrin complex to facilitate iron into cells. TFR1 is ubiquitously expressed on the surfaces of generic cells, whereas TFR2 is specially expressed in liver cells. TFR1 has attracted more attention than TFR2 by having diverse functions in both invertebrates and vertebrates. Recently reports showed that TFR1 involved in many kinds of diseases including anemia, neurodegenerative diseases and cancers. Most importantly, TFR1 has been verified to be abnormally expressed in various cancers. Thus, TFR1 is postulated as a potential molecular target for diagnosis and treatment for cancer therapy.

In one embodiment, at least one of the ligand types, and preferably one ligand type, targets folate receptor 1 (FOLR1). The protein encoded by this gene is a member of the folate receptor family. Members of this gene family bind folic acid and its reduced derivatives, and transport 5-methyltetrahydrofolate into cells. This gene product is a secreted protein that either anchors to membranes via a glycosyl-phosphatidylinositol linkage or exists in a soluble form. Mutations in this gene have been associated with neurodegeneration due to cerebral folate transport deficiency.

The folate cycle sustains key metabolic reactions and is essential for rapidly growing cells. Under physiologic conditions, exogenous reduced folates (water-soluble B vitamins) are predominantly transported into cells via the low-affinity, high-capacity, ubiquitously expressed reduced folate carrier (RFC; bidirectional anion-exchange mechanism). Once in the cell, folates play an essential role in the biosynthesis of purines and thymidine, which in turn are required for DNA synthesis, methylation, and repair. Folates are also transported by high-affinity FRs. In humans, there are four isoforms of the FR (FRα, FRβ, FRγ, and FRδ). FRα, FRβ, and FRδ are attached to the cell surface by a glycosylphosphatidylinositol anchor, while FRγ is a secreted protein. Because FRα is expressed on the cell surface in a tumour-specific manner, it provides the potential to allow not only tumour localization, but also selected delivery of therapeutic agents to the malignant tissue, minimizing collateral toxic side-effects.

There are a number of unique advantages to exploiting FR as a diagnostic and therapeutic target. First, FRα is located on the luminal surface of epithelial cells in most proliferating nontumor tissues and is inaccessible to circulation. In contrast, FRα is expressed all over the cell in malignant tissue and is accessible via circulation. Second, FR has the ability to bind to folic acid, a relatively innocuous, small molecule that can rapidly penetrate solid tumours and is amenable to chemical conjugation with other molecules. Once a folate conjugate is bound to FR, it is internalized into the cell and the FRα is rapidly recycled to the cell surface via the FR-mediated endocytic pathway. These factors all emphasize the potential role of FRα in the diagnosis and treatment of specific tumour types.

In one embodiment, at least one of the ligand types, and preferably one ligand type, targets epidermal growth factor receptor (EGFR). The protein encoded by this gene is a transmembrane glycoprotein that is a member of the protein kinase superfamily. This protein is a receptor for members of the epidermal growth factor family. EGFR is a cell surface protein that binds to epidermal growth factor. Binding of the protein to a ligand induces receptor dimerization and tyrosine autophosphorylation and leads to cell proliferation.

Epidermal growth factor receptors (EGFRs) are a large family of receptor tyrosine kinases (TK) expressed in several types of cancer, including breast, lung, esophageal, and head and neck. EGFR and its family members are the major contributors of a complex signaling cascade that modulates growth, signaling, differentiation, adhesion, migration and survival of cancer cells. EGFR binds to its cognate ligand EGF, which further induces tyrosine phosphorylation and receptor dimerization with other family members leading to enhanced uncontrolled proliferation. Due to their multi-dimensional role in the progression of cancer, EGFR and its family members have emerged as attractive candidates for anti-cancer therapy. Specifically, the aberrant activity of EGFR has shown to play a key role in the development and growth of tumor cells, where it is involved in numerous cellular responses including proliferation and apoptosis. The epidermal growth factor receptor (EGFR) signalling pathway is also a strong contender for both initiating and determining clinical outcomes in many respiratory diseases. Deregulation of the EGFR pathway causing aberrant EGFR signalling is associated with the early stage pathogenesis of lung fibrosis, cancer and numerous airway hypersecretory diseases, including COPD, asthma and cystic fibrosis.

Ligands for binding to each of these receptor are well known in the art. Example ligands for LRP-1 and SCARB1 are discussed above. Example ligands for TFRCs, e.g. TFR1, are transferrin and transferrin mimic peptide. An example ligand for FOLR1 is folic acid. An example ligand for EGFR is the peptide YHWYGYTPQNVI peptide.

A further example of a ligand is a ligand that is adapted to enable the polymersome to bind to an immune cell. Illustrative and non-limiting examples of such ligands include phosphorylcholine (as discussed in more detail below), peptidoglycan, lipoproteins, glycolipids, lipopolysaccharide, lipopeptides, synthetic compounds such as loxoribine and bropirimine, peptidoglycans, acetylated/malelylated proteins, modified low-density lipoproteins, polyanionic ligands, sulfated sugars, mannose-modified polysaccharides, fucose-modified polysaccharides, galactose-modified polysaccharides, proteins and β-glucan. In immune system cells targeting, the specific and precise targeting requires a particularly high level of discrimination/precision, which can be afforded by the polymersomes of the present invention.

Targeting of immune cells is believed to be important in treating immune-related diseases, such as autoimmune diseases and graft rejection, and for improving preventive/therapeutic vaccines. The cell membrane provides a remarkable example of spatiotemporal control of complex biological interactions thanks to hundreds of different ligands-receptors interactions selected trough evolution with the right amount of affinity and multi-combinatorial binding. Targeting of immune cells may also be important in embodiments where the nucleic acid cargo is an mRNA, and the polymersome forms part of an mRNA vaccine which is devised to induce production of an antigen in vivo by the subject to whom the polymersome is administered.

Therapies that target components of the defence system such as neutrophils and neutrophil-associated effectors are promising for adjunct host-directed therapies to improve antibiotic efficacy, i.e. in tuberculosis treatment, and reduce both treatment time and long-term pathological sequelae. Neutrophils have however proven very difficult cells to manipulate, and to the knowledge of the inventors, no commercially available vector exists that enables the efficient intracellular delivery of cargo within their short life span without compromising their viability and activation state. Expression of high levels of immune cells including neutrophils has been associated with detrimental outcome in several solid tumours and new strategies to decrease their presence and activity are currently under clinical development. Accordingly, neutrophils are desirable targets for the polymersomes of the present invention.

A ligand can be attached to the external surface of the polymersome using routine techniques, for example by adapting well known methods for attaching ligands to polymers, drugs, nucleic acids, antibodies and other substances. The attachment may be non-covalent (e.g. electrostatic) or covalent, though it is preferably covalent. For example, the targeting moiety can be attached by reacting a suitable functional group on the targeting moiety (including but not limited to an amine group, a carboxyl group and a thiol group) with a corresponding functional group on at least one of the copolymers that form, or will form, the polymersome. The attachment can be effected either before the polymersome structure is formed from the copolymers, or after the polymersomes have been formed.

In a particular embodiment, the polymersome comprises, on its external surface, a polymer brush comprising poly(ethylene glycol)-poly(2-(diisopropylamino)ethyl methacrylate) and each ligand type. Thus, the ligands are inserted in the polymer brush of polymersomes made of poly(ethylene glycol)-poly(2-(diisopropylamino)ethyl methacrylate), typically by employing a solvent-switch method. Typically, the density of the ligands within the brush can also be varied.

It is also possible to provide for attachment of the ligand to the copolymers by first chemically activating either or both of the ligand and the copolymers. For example, a peptide ligand may be activated by adding a reactive species to one of its termini, such as a cysteine moiety (whose thiol group is well known to react readily with functional groups such as the widely used maleimide moiety). Similarly, a copolymer can be activated by functionalising it with a reactive species (e.g. a maleimide moiety when the targeting moiety carries a thiol group). The copolymer may be provided with such a reactive species either by functionalisation of the copolymer itself, or by providing suitable monomers prior to the polymerisation that forms the copolymer, or by providing a suitable initiator for the polymerisation.

In an embodiment, the polymersome comprises one or more ligands on its external surface of the polymersome that are covalently bound to a poly(ethylene glycol) molecule. Tethering of the ligands to PEG molecules of different chain lengths in this way enables control over the deepness of the ligand insertion within the polymer brush. This in turn affects the steric repulsive potential, Eₛ, between the ligand and the target cell surface receptor. As discussed above, this steric potential is an important factor in determining the optimum number of ligands on the surface of the polymersome for binding to a particular cell type.

A ligand may be attached directly to the external surface of the polymersome, or alternatively it may be attached via a chemical spacer. A ligand may also be a pendant group of a polymer comprised by the polymersome (i.e. at least one of the copolymers forming the polymersome itself). Clearly in this embodiment it is not necessary to undertake separate synthetic steps to attach the ligand to the copolymer or the resulting polymersome. Suitable pendant groups generally include any group that corresponds to a ligand as defined elsewhere herein. In one illustrative embodiment, the targeting moiety is a phosphorylcholine moiety, i.e. a group having the formula

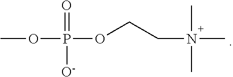

A phosphorylcholine moiety is a zwitterionic moiety that can constitute a pendant group in one or more of the monomers that form the copolymers comprised in a polymersome.

The phosphorylcholine moiety selectively targets scavenger receptor class B, member 1 (SCARB1) over-expressed by macrophages and other immune cells; in particular it enables a polymersome featuring phosphorylcholine moieties to enter such cells. Thus, polymersomes featuring a phosphorylcholine targeting moiety are particularly suitable for use in the treatment of inflammatory and/or immune disorders.

### Encapsulated Drug

The cargo-containing polymersome of the present invention may comprise a drug encapsulated within the polymersome, in addition to the nucleic acid/nucleic acid-binding protein cargo. For the avoidance of doubt, it is also possible to encapsulate a plurality of different such drugs within a single polymersome, or to provide a plurality of polymersomes each encapsulating a particular such drug.

As will be readily understood, the encapsulated drug is selected in accordance with the disorder to be treated. Non-limiting examples of such disorders are described elsewhere in this disclosure.

Non-limiting examples of drugs include: a drug that is effective for the treatment or prevention of a brain disorder; a drug that is effective for the treatment or prevention of the immune and/or inflammatory disorder; and a drug that is effective for the treatment or prevention of a cancer. There is no particular limitation on the identity of the drug and so drugs can be selected from those known in the art for treatment or prevention of the disorder of interest in any given embodiment.

Non-limiting examples of drugs include neuroprotectants, immunomodulatory drugs (“immunomodulators”), non-steroidal anti-inflammatory drugs (NSAIDs), corticosteroids, disease-modifying antirheumatic drugs (DMARDs,) immunosuppressants, TNF-alpha inhibitors and anti-cancer drugs.

Illustrative and non-limiting examples of specific drugs that may be encapsulated include fumarate and fumarate esters, glutamate antagonists (e.g., Estrogen, Ginsenoside Rd, Progesterone, Simvastatin, Memantine), antioxidants (e.g., Acetylcysteine, Crocin, Fish oil, Minocycline, Pyrroloquinoline quinone (PQQ), Resveratrol, Vinpocetine, Vitamin E), Stimulants (e.g., Selegiline, Nicotine, Caffeine), Caspase inhibitors, Trophic factors (e.g., CNTF, IGF-1, VEGF, and BDNF), Anti protein aggregation agents (e.g. sodium 4-phenylbutyrate, trehalose, and polyQ-binding peptide), Erythropoietin, Lithium, carnosine, asiatic acid, flavonoids (e.g. xanthohumol, naringenin, galangin, fisetin and baicalin), cannabinoids (e.g., WIN55,212-2, JWH-133 and TAK-937), citicoline, minocycline, cerebrolysin, ginsenosoid-Rd, granulocyte-colony stimulating factor, Tat-NR2B9c, magnesium, albumin, paracetamol, aspirin, choline and magnesium salicylates, celecoxib, diclofenac (e.g. diclofenac potassium, diclofenac sodium), diflunisal, etodolac, fenoprofen, flurbiprofen, ibuprofen, indomethacin, ketoprofen, meclofenamate, mefenamic acid, meloxicam, nabumetone, naproxen (including naproxen sodium), oxaprozin, piroxicam, rofecoxib, salsalate, sodium salicylate, sulindac, tolmetin, valdecoxib, corticosteroids, alemtuzumab, interferon beta-1b, fingolimod, glatiramer acetate, natalizumab, plegridy, peginterferon beta 1a, teriflunomide, methotrexate, sulfasalazine, leflunomide, adalimumab, etanercept, golimumab, ustekinumab, azathioprine, cyclosporine, infliximab, golimumab, certolizumab, hydroxychloroquine, methotrexate, azathioprine, mycophenolate, acitretin, hydrea, isotretinoin, mycophenolate mofetil, sulfasalazine, 6-thioguanine, calcipotriol, calcitriol, tacalcitol, tacrolimus, pimecrolimus, dithranol, endamustine, bendamustine, carmustine, chlorambucil, cyclophosphamide, dacarbazine, ifosfamide, melphalan, procarbazine, streptozocin, temozolomide, capecitabine, 5-Fluoro Uracil, Fludarabine, Gemcitabin, Methotrexate, Pemetrexed, Raltitrexed, Actinomycin D, Bleomycin, Doxorubicin, Epirubicin, Mitomycin, Mitoxantrone, Etoposide, Docetaxel, Irinotecan, Paclitaxel, Topotecan, Vinblastine, Vincristine, Vinorelbine, Eribulin, Carboplatin, Cisplatin, Oxaliplatin, Afatinib, Aflibercept, BCG, Bevacizumab, Brentuximab, Cetuximab, Crizotinib, Denosumab, Erlotinib, Gefitinib, Imatinib, Interferon, Ipilimumab, Lapatinib, Panitumumab, Pertuzumab, Rituximab, Sunitinib, Sorafenib, Trastuzumab emtansine, Temsirolimus, Trastuzumab, Vemurafenib, Clodronate, Ibandronic acid, Pamidronate, Zolendronic acid, Anastrozole, Abiraterone, Bexarotene, Bicalutamide, Buserelin, Cyproterone, Degarelix, Exemestane, Flutamide, Folinic acid, Fulvestrant, Goserelin, Lanreotide, Lenalidomide, Letrozole, Leuprorelin, Medroxyprogesterone, Megestrol, Mesna, Octreotide, Stilboestrol, Tamoxifen and Thalidomide.

### Pharmaceutical Compositions

The polymersome of the present invention can be formulated as a pharmaceutical composition using routine techniques known in the art.

The pharmaceutical composition comprises a plurality of the polymersomes of the present invention. It also comprises one or more pharmaceutically acceptable excipients or diluents. The one or more pharmaceutically acceptable excipients or diluents may be any suitable excipients or diluents. The pharmaceutical composition is typically aqueous, i.e. it contains water (in particular sterile water).

A typical pH of the aqueous pharmaceutical composition is 7.0 to 7.6, preferably 7.2 to 7.4. Pharmaceutically acceptable buffers may be used to achieve the required pH. The pharmaceutical composition may be in the form of a sterile, aqueous, isotonic saline solutions.

Typically the pharmaceutical composition is an injectable composition, e.g. it is suitable for intravenous delivery, for example it is suitable for infusion.

The present invention also extends to hydrogels comprising a plurality of polymersomes of the invention. Such hydrogels may comprise entangled worm-like micelles, which can trap water, together with other substances of interest. The hydrogels can be used without limitation for any known application of hydrogels, including but by no means limited to preparation of delivery agents, as adjuvants in vaccines, and in tissue engineering applications.

### Medical Uses of the Polymersomes

The polymersomes and pharmaceutical formulations of the present invention are able to target tissues including, but not limited to cells (e.g. CNS cells) beyond the blood-brain barrier, immune cells and cancer cells and to release the nucleic acid/nucleic acid-binding protein (and any further encapsulated drugs) once localised at the target. As discussed above, the polymersomes may comprise ligands on their external surface (e.g. as part of the polymers themselves or as distinct moieties attached thereto) which results in a high targeting efficiency for the target cell type.

As will be readily understood, the nucleic acid cargo, any further encapsulated drug, and any ligand present on the external surface of the polymersome are selected in accordance with the disease to be treated. For example, if the disorder is a cancer then the nucleic acid may be a nucleic acid which induces gene expression, gene knockdown or RNAi interference in such a way that disrupts the cell signaling pathways involved in growth of the cancer, a further encapsulated drug that is effective for the treatment or prevention of the cancer, and the polymersome may comprise ligands on its external surface that are adapted to enable the polymersome to bind to (and typically enter) a cancer cell.

Examples of cancers include: cancers of the skin, such as melanoma; lymph node; breast; cervix; uterus; gastrointestinal tract; lung; ovary; prostate; colon; rectum; mouth; brain; head and neck; throat; testes; thyroid; kidney; pancreas; bone; spleen; liver; bladder; larynx; nasal passages; AIDS-related cancers; cancers of the blood and bone marrow, such as multiple myeloma and acute and chronic leukemias, for example, lymphoblastic, myelogenous, lymphocytic, and myelocytic leukemias; advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, chronic lymphocytic leukemia (CLL), Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma (localized melanoma, including, but not limited to, ocular melanoma), malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, eiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

The polymersomes of the present invention may also be useful in the treatment of infectious disease. Examples of infectious diseases include: Acinetobacter infections, Actinomycosis, Adenovirus infection, African sleeping sickness (African trypanosomiasis), AIDS (acquired immunodeficiency syndrome), Amoebiasis, Anaplasmosis, Angiostrongyliasis, Anisakiasis, Anthrax, Arcanobacterium haemolyticum infection, Argentine hemorrhagic fever, Ascariasis, Aspergillosis, Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Dental caries, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Coxsackie B virus infection, Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Eastern equine encephalitis (EEE), Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Human T-lymphotropic virus 1 infection, Hymenolepiasis, Epstein-Barr virus infectious mononucleosis (Mono), Influenza (flu), Isosporiasis, Japanese encephalitis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus, (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, Severe acute respiratory syndrome (SARS), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tick-borne encephalitis, Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxic shock syndrome (TSS), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever and Zygomycosis.

Further disorders that may be susceptible to treatment or prevention with the polymersomes of the invention include brain disorders, inflammatory or autoimmune diseases, atherosclerosis, ischemic heart disease, liver disorders, kidney disorders, diseases associated with ageing, and genetic diseases.

In one embodiment, the disorder that may be susceptible to treatment or prevention with the polymersomes of the invention is a genetic disease. The genetic disease may be selected from 1p36 deletion syndrome, 1q21.1 deletion syndrome, 2q37 deletion syndrome, 5q deletion syndrome, 5,10-methenyltetrahydrofolate synthetase deficiency, 17q12 microdeletion syndrome, 17q12 microduplication syndrome, 18p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, Absence deformity of leg-cataract syndrome, Aceruloplasminemia, Acheiropodia, Achondrogenesis type II, achondroplasia, Acute intermittent porphyria, Adenylosuccinate lyase deficiency, Adrenoleukodystrophy, Alagille syndrome, ADULT syndrome, Aicardi-Goutieres syndrome, Albinism, Alexander disease, Alfi's syndrome, alkaptonuria, Alport syndrome, Alternating hemiplegia of childhood, Aortic arch anomaly—peculiar facies—intellectual disability, Amish lethal microcephaly, Amyotrophic lateral sclerosis—Frontotemporal dementia, Angel-shaped phalango-epiphyseal dysplasia, Alström syndrome, Alzheimer's disease, Amelogenesis imperfecta, Aminolevulinic acid dehydratase deficiency porphyria, Androgen insensitivity syndrome, Angelman syndrome, Aphalangy-syndactyly-microcephaly syndrome, Apert syndrome, Arthrogryposis-renal dysfunction-cholestasis syndrome, Ataxia telangiectasia, Axenfeld syndrome, Bainbridge-Ropers syndrome, Beare-Stevenson cutis gyrata syndrome, Beckwith-Wiedemann syndrome, Benjamin syndrome, biotinidase deficiency, Bjornstad syndrome, Blepharophimosis intellectual disability syndromes, Bloom syndrome, Birt-Hogg-Dube syndrome, Brody myopathy, Brunner syndrome, CADASIL syndrome, Cat eye syndrome, CRASIL syndrome, Chronic granulomatous disorder, Campomelic dysplasia, Camptodactyly-taurinuria syndrome, Canavan disease, Carpenter Syndrome, CDKL5 deficiency disorder, Cerebral dysgenesis-neuropathy-ichthyosis-keratoderma syndrome (CEDNIK), Cleft palate short stature vertebral anomalies syndrome, Combined malonic and methylmalonic aciduria (CMAMMA), Combined malonic and methylmalonic aciduria (CMAMMA), Congenital muscular dystrophy-infantile cataract-hypogonadism syndrome, Cystic fibrosis, Charcot-Marie-Tooth disease, CHARGE syndrome, Chediak-Higashi syndrome, Chondrodysplasia, Grebe type, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Corneal dystrophy-perceptive deafness syndrome, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Currarino syndrome, Darier's disease, Dent's disease (Genetic hypercalciuria), Denys-Drash syndrome, De Grouchy syndrome, Dolichonychia, Down Syndrome, DiGeorge syndrome, Distal hereditary motor neuropathies, multiple types, Distal muscular dystrophy, Duchenne muscular dystrophy, Dravet syndrome, Ectrodactyly-polydactyly syndrome, Edwards Syndrome, Ehlers-Danlos syndrome, Emanuel syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Familial episodic pain syndrome, Familial thoracic aortic aneurysm and aortic dissection, Feingold syndrome, FG syndrome, FBXW7 neurodevelopmental syndrome, Fibular aplasia-ectrodactyly syndrome, Fine-Lubinsky syndrome, Fragile X syndrome, Friedreich's ataxia, G6PD deficiency, Galactosemia, Gaucher disease, Gerstmann-Straussler-Scheinker syndrome, Gillespie syndrome, Glutaric aciduria, type I and type 2, GRACILE syndrome, GRIN2B-related neurodevelopmental disorder, Griscelli syndrome, Gustavson syndrome, Hailey-Hailey disease, Harlequin type ichthyosis, Hemochromatosis type 1, Hemochromatosis type 2A, Hemochromatosis type 2B, Haemochromatosis type 3, Hemochromatosis type 4, Hemochromatosis type 5, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Infantile cerebral and cerebellar atrophy with postnatal progressive microcephaly, Ischiopatellar dysplasia, Isodicentric 15, PRICKLE1-related progressive myoclonus epilepsy with ataxia, Jackson-Weiss syndrome, Jacobsen syndrome, Joubert syndrome, Juvenile-onset dystonia, Juvenile primary lateral sclerosis (JPLS), Keloid disorder, KIF1A-Associated Neurological Disorder, Kleefstra syndrome, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, familial, Menkes disease, Methemoglobinemia, Methylmalonic acidemia, Micro syndrome, Microcephaly, Miller-Dieker syndrome, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, NEDAMSS (neurodevelopmental disorder with regression, abnormal movements, loss of speech and seizures), Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Ostravik-Lindemann-Solberg syndrome, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phelan-McDermid syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Proximal 18q deletion syndrome, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Scheuermann's disease, Schwartz-Jampel syndrome, Sjogren-Larsson syndrome, Skin fragility-woolly hair-palmoplantar keratoderma syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), Split hand split foot-nystagmus syndrome, SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Thickened earlobes-conductive deafness syndrome, Treacher Collins syndrome, Tuberous sclerosis complex (TSC), Turner syndrome, Usher syndrome, Variegate porphyria, Viljoen-Kallis-Voges syndrome, von Hippel-Lindau disease, von Willebrand disease, Waardenburg syndrome, Warkany syndrome 2, Weissenbacher-Zweymuller syndrome, Weyer's ulnar ray/oligodactyly syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (triple X syndrome), XXXX syndrome (48, XXXX), XXXXX syndrome (49,XXXXX), XXXXY syndrome (49,XXXXY), XYY syndrome (47,XYY), XXYY syndrome (48,XXYY), XYYY syndrome (48,XYYY), XXXY syndrome (48,XXXY), XYYYY syndrome (49,XYYYY) and Zellweger syndrome.

Medical uses and methods of treatment, of course, involve the administration of a therapeutically effective amount of the polymersome. A therapeutically effective amount of the polymersomes is administered to a patient. A typical dose is from 0.001 to 1000 mg, measured as a weight of the drug, according to the activity of the specific drug, the age, weight and conditions of the subject to be treated, the type and severity of the disease and the frequency and route of administration. Preferably, daily dosage levels are from 0.001 mg to 4000 mg.

The present invention further provides a method of treating or preventing a disorder that comprises administering a therapeutically effective amount of a nanoparticle or microparticle of the invention to a patient in need thereof. For example, the present invention provides a method of treating or preventing a disorder selected from any disorder specified in this disclosure, the drug being a drug that is capable of treating or preventing the said disorder, such as a brain disorder, an immune and/or inflammatory disorder, a cancer, or a genetic disease. The present invention still further provides the use of a nanoparticle or microparticle of the present invention in the manufacture of a medicament for use in a method of treating or preventing a disorder as identified above.

The present invention further provides a vaccine comprising a cargo-containing polymersome according to the invention, and one or more pharmaceutically acceptable excipients or diluents. In a particularly preferred embodiment, the cargo within the polymersome comprises an mRNA that encodes an antigen, and an mRNA-binding protein. An antigen is any agent that causes the immune system of an animal body to produce an immune response, e.g. bacteria, viruses or pollen. Typically, after administration of the vaccine to a human or animal recipient, the human or animal recipient transcribes the mRNA within the vaccine to produce the antigen. The production of this antigen in vivo induces the memory function of the adaptive immune system towards the specific antigen. Preferably, the vaccine is a vaccine against infectious disease (e.g. HIV, influenza, or a coronavirus such as the common cold or COVID-19), or a cancer vaccine.

In all aspects of the present invention, the polymersomes may further comprise a label or imaging agent (e.g., encapsulated therein and/or attached to the surface of the particles). For instance, the label/imaging agent could be a dye. A dye for imaging refers to any substance that is used as a label, or that enhances specific structures in any imaging technique. An imaging agent, hence, includes optical an imaging agent, magnetic resonance imaging agent, radioisotope, and contrast agent. Examples, without limitation, of optical imaging agents are an acridine dye, a coumarin dye, a rhodamine dye, a xanthene dye, a cyanine dye, a pyrene dye, Texas Red, Alexa Fluor® dye, BODIPY® DYE, Fluorescein, Oregon Green® dye, and Rhodamine Green™ dye, which are commercially available or readily prepared by methods known to those skilled in the art. Examples of imaging agents appropriate for the present invention include, but are not limited to, transition metals and radioactive transition metals chelated to chelating agents for instance DTPA (diethylene triamine pentaacetic adic), DOTA (1,4,7,10-tetraazacyclododeane-1,4,7-tetraacetic acid) and NOTA (1,4,7-Triazacyclononane-1,4,7-triacetic acid).

Further, in all aspects of the present invention, the polymersomes may further comprise a targeting unit, i.e. antibodies, peptides, proteins etc. (e.g. encapsulated therein and/or attached to the surface of the particles). A targeting unit is any chemical structure that functionally interacts with a binding site to cause a physical association between the agent and a surface, e.g., a cell surface. The term targeting unit embraces any molecule (e.g. a naturally occurring molecule, or a chemically/physically modified variant thereof) that is capable of binding to a binding site on the target surface. The binding site could be, but not exclusively, also be capable of internalisation (e.g. endosome formation), also referred to as receptor-mediated endocytosis. The targeting unit may possess an endosomal membrane translocation function, in which case separate targeting unit and translocation domain components need not be present in an agent of the present invention.

### EXAMPLES

The present invention is illustrated by the following examples. However, these examples do not limit the scope of the invention.

### Example 1: Formulation of Polymersomes

Polymersomes comprising both (i) free nucleic acid cargo and (ii) a cargo comprising a nucleic acid and a nucleic acid-binding protein were formulated.

Synthetic polymersome vesicles were made using amphiphilic copolymers comprising either poly(ethylene glycol) (PEG) as a hydrophilic block and poly(ethylene glycol)-poly(2-(diisopropylamino)ethyl methacrylate) (PEG-PDPA) as a hydrophilic block, or poly(ethylene glycol) (PEG) as a hydrophilic block and poly(lactic acid) (PEG-PLA) as a hydrophilic block. Four polymersomes were prepared in total:
(1) Polymersomes comprising PEG-PLA copolymers and a cargo of free pcDNA3.1NL, a plasmid DNA (pDNA) which encodes for the NanoLuc luciferase protein;
(2) Polymersomes comprising PEG-PLA copolymers and a cargo comprising pcDNA3.1NL and the histone protein H1;
(3) Polymersomes comprising PEG-PDPA copolymers and a cargo of free pcDNA3.1NL; and
(4) Polymersomes comprising PEG-PDPA copolymers and a cargo comprising pcDNA3.1NL and the histone protein H1.

### Example Synthesis of PEG-PDPA

Poly(ethylene glycol)-poly(2-(diisopropylamino)ethyl methacrylate) (PEG-PDPA) was synthesized by atom transfer radical polymerization (ATRP), where the monomer 2-(diisopropylamino)ethyl methacrylate (DPA) and the initiator PEG-bound 2-bromo-2-methylpropanoate (PEG-Br) were dissolved in degassed isopropanol with copper(I) bromide (CuBr) and 2,2′-bipyridyl as the catalyst. The polymer was purified by silica column and dialysis and obtained in powder form after lyophilization.

### Example Synthesis of PEG-PLA

Poly(ethylene glycol)-poly(D,L-lactic acid) (PEG-PLA) was synthesized by ring-opening polymerization (ROP) of D,L-lactide, initiated from hydroxyl-ended PEG in dichloromethane with 1,8-Diazabicyclo[5.4.0]undec-7-ene (DBU) as the catalyst. The polymer was purified by dialysis and obtained in powder form after lyophilization.

### Formulation of Polymersomes Comprising Nucleic Acid Cargo:

A solvent switch method was used for PEG-PDPA to form polymersomes. PEG-PDPA was solubilized in THE (in some embodiments, PEG-PDPA functionalized with ligands can be additionally solubilized in DMSO, and subsequently added to the solution of non-functionalized PEG-PDPA in THF). Subsequently water was injected into the polymer-containing organic solution to promote polymersome formulation. After purification with dialysis the obtained polymersomes were divided into two vials and (i) mixed with a nucleic acid and electroporated, or (ii) mixed with a nucleic acid complexed with histone H1 and electroporated. The samples were purified from the unloaded nucleic acids and stored at 4° C.

A solvent displacement method was used for PEG-PLA. A solution of PEG-PLA was prepared in DMF, and subsequently this solution was added to water using a syringe pump to promote polymersome formation. The nucleic acid with and without histone H1 was added during the self-assembly of PEG-PLA polymersomes. After purification with dialysis the obtained nucleic acid loaded polymersomes were purified from the unloaded nucleic acids and stored at 4° C.

The particle size distribution of the polymersomes was measured via dynamic light scattering (DLS). A Malvern Zetasizer Nano ZS laser light scatterer equipped with a He—Ne 4 mW 633 nm laser was used. Polymersomes were diluted in filtered milliQ water in 1 mL disposable cuvettes, and experiments were an average of n=3 runs at a set angle of 1730.

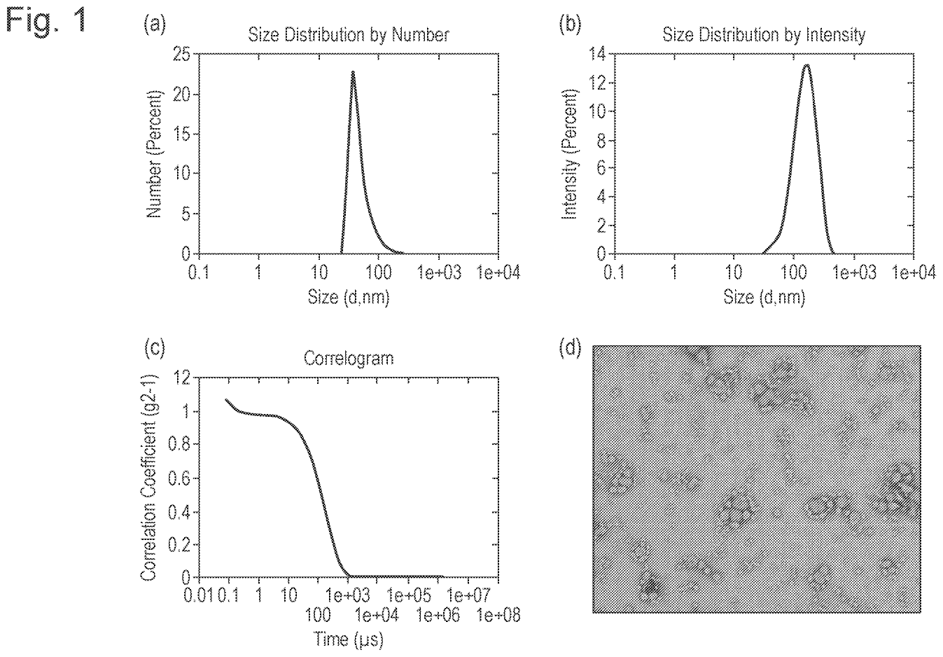

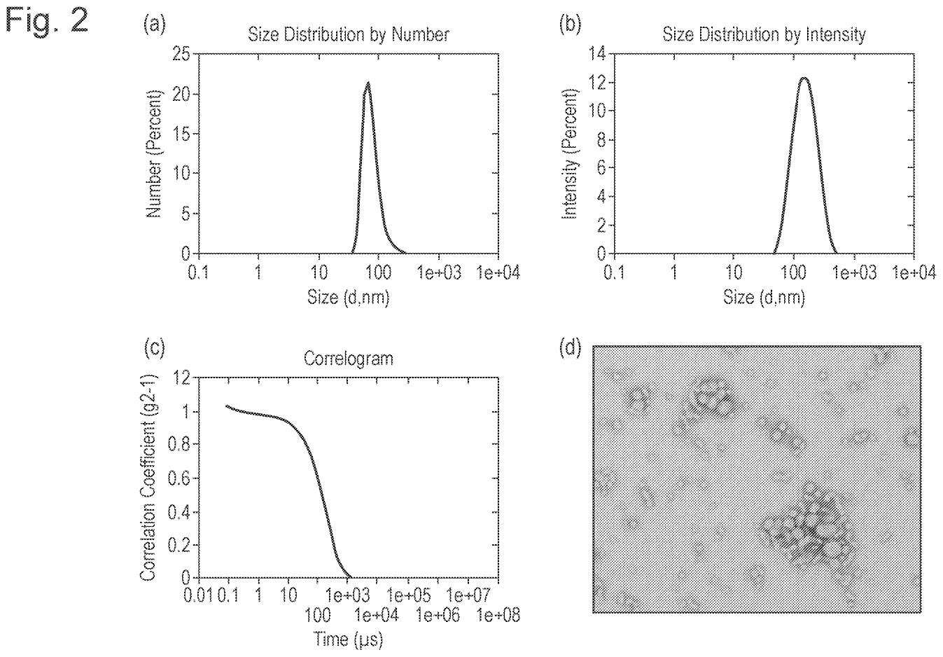

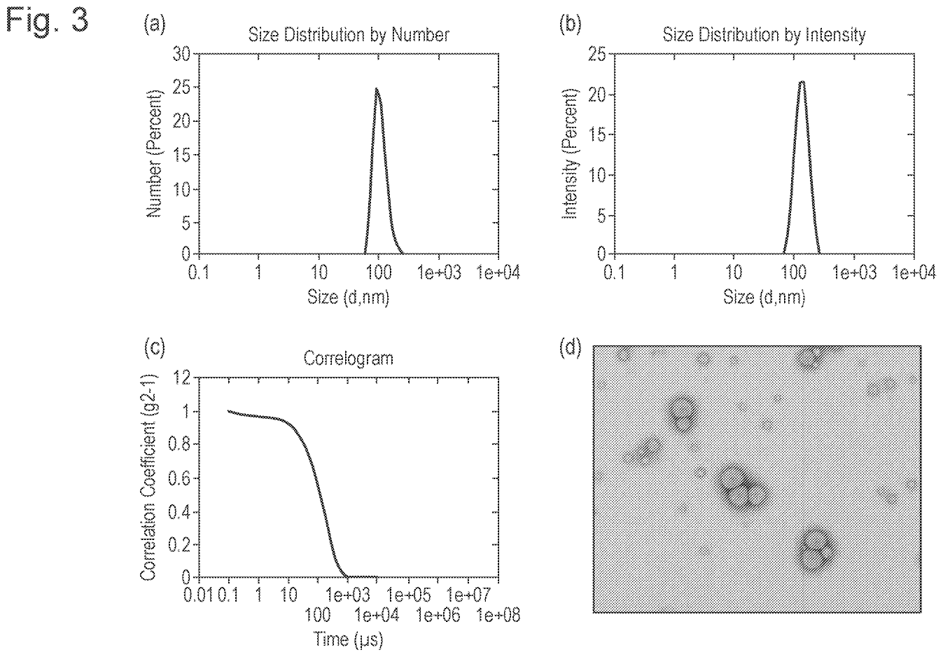

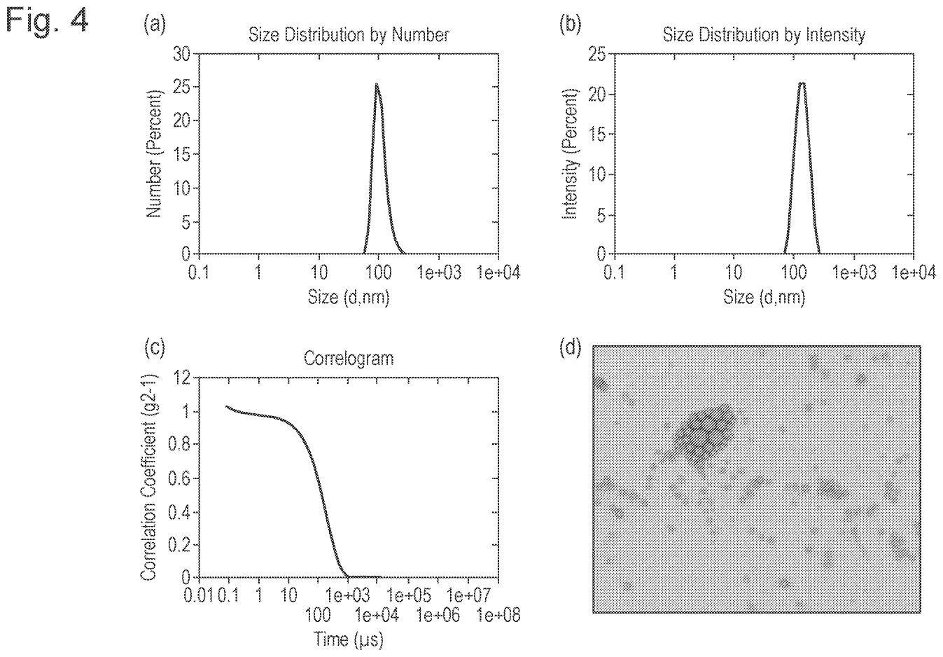

The results of the DLS experiments are shown in FIGS. 1-4 for polymersomes (1) to (4), respectively. The DLS measurements suggest the formation of spherical structures. This feature implies that the copolymers assemble into membranes that in turn form into spherical polymersomes. FIGS. 1(a), 2(a), 3(a) and 4(a) show the particle size distributions by number, whilst FIGS. 1(b), 2(b), 3(b) and 4(b) show the particle size distributions by intensity. These distributions demonstrate that all the polymersomes have an average diameter of approximately 100 nm, which is considered to be a desirable particle size for in vivo applications of the polymersomes. The choice of copolymer (PEG-PDPA or PEG-PLA) or the choice of cargo (with or without nucleic acid-binding protein) does not affect the particle size distribution. In FIGS. 1(c), 2(c), 3(c) and 4(c) a correlogram of the DLS data is plotted. Dynamic light scattering measures the correlation coefficient from intensity traces performed using an integrated digital correlator. As can been seen the measured correlation curves have all similar decayed time to baseline, confirming that the size of the polymersomes in solution in all the samples is very similar.

Polymersomes in filtered milliQ water were also assessed for morphology using transmission electron microscopy (TEM). Samples were mounted on glow-discharged carbon coated grids by submerging the grids into the polymersome solution for 60 seconds, followed by staining for 5 seconds using 0.5% (w/w) phosphotungstic acid (PTA) and dried under vacuum and assessed via a JEOL microscope using 100 kV voltage tension.

The resulting TEM micrographs are shown (for each polymersome sample, respectively) in FIGS. 1(d), 2(d), 3(d) and 4(d). Transmission electron microscopy (TEM) was used to confirm the vesicular structure. All the samples appeared spherical, with varied diameters, in agreement with DLS measurements, and moreover support vesicle formation.

### Example 2: Delivery of Polymersomes to Cells

The ability of the synthesized polymersomes comprising a free pDNA cargo (pcDNA3.1NL) to be internalized within cells was measured in vitro using the human embryonic kidney cell line HEK293T (purchased from ATCC). The pDNA cargo was covalently bound to the fluorescent reporter label Cy5 (a far-red-fluorescent cyanine dye). HEK293T cells were cultured in DMEM supplemented with 10% FBS, 1% Pyruvate and 1% Penicillin/Streptomycin Cells were treated for 48 hours with either (a) 0.5 mg/mL PEG-PLA polymersomes containing free pcDNA3. 1NL tagged with Cy5 as cargo, (b) 0.5 mg/mL PEG-PDPA polymersomes containing free pcDNA3.1NL tagged with Cy5 as cargo, or (c) pcDNA3.1NL tagged with Cy5 complexed with the transfection reagent JetOPTIMUS® (purchased from PolyPlus transfection), used as a positive control.

The uptake of the Cy5-labelled pDNA into the HEK293T cells was monitored via flow cytometry.

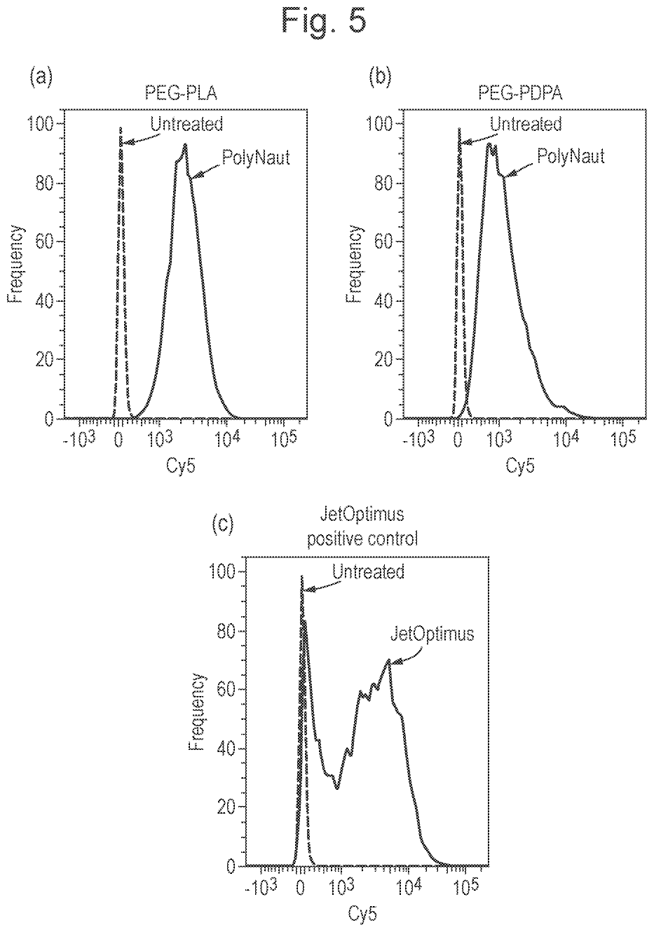

The results are shown in FIG. 5. In each graph (FIGS. 5(a), 5(b) and 5(c)) the “untreated” peak represents the negative control experiment, showing that in the absence of any applied Cy5-labelled pcDNA, cells have a unimodal fluorescence intensity distribution around a peak defined as ‘background’ or intrinsic cell fluorescence intensity. The unimodal fluorescence distribution with a peak at around 10³ fluorescence units that is observed in FIGS. 5(a) and 5(b) for the “treated” cells shows that the polymersome-encapsulated pDNA cargo is efficiently internalized in the HEK293T cells with uniform delivery. The bimodal fluorescence distribution observed in FIG. 5(c) for the positive control shows that there is a significant proportion of cells in which no Cy5-labelled pDNA was internalized (the left-hand peak for the treated cells at low fluorescence intensity), as well as a proportion of cells in which the Cy5-labelled pDNA is internalized (the right-hand peak for the treated cells at high fluorescence intensity).

Accordingly, it is observed that the polymersomes described herein result in an improved internalization of nucleic acid cargo into cells, compared with standard transfection reagents such as JetOPTIUS®. Substantially all the cells treated with the polymersomes as described herein internalized the nucleic acid cargo with a high level of consistency.

### Example 3: Gene Expression In Vitro

Next, the effect of the presence of the nucleic acid-binding protein within the polymersome on the efficiency of pDNA gene expression was investigated.

HEK293T cells were cultured as described in Example 2, and were treated with polymersome (0.5 mg/mL in PBS) compositions (1) to (4) as prepared in Example 1. As a negative control, a sample of untreated HEK293T cells were also incubated under the same conditions. After incubation for 48 hours, the NanoLuc luciferase expression was measured using a NanoLuc activity assay, as follows:

On day 1 the cells were seeded in a 96-well black plate with and incubated at 37° C. The following day the cells were treated with (i) JetOPTIMUS transfection kit at final pDNA concentration of 0.02 ng/uL, (ii) polymersome-formulated pDNA (i.e. each of polymersome formulations (1) to (4)) at 1 ng/μl. The samples were incubated for 48 hours at 37° C. prior to readout by multiplexed CellTiter-Blue:NanoLuciferase assay (Promega).

Here cells were incubated with ⅕ CellTiter-Blue for 4 hours in DMEM growth media, cellular conversion of resazurin to resorufin was measured by fluorescent plate reader as a metric of cell number per well. After assaying CellTiter-Blue, cells were equilibrated to room temperature and treated with freshly prepared 1:50 NanoGlo Buffer:Reagent solution. Luminscence was measured per well using a TECAN Spark plate reader with 1 second integration time. All the results have been expressed as ratio of Luminscence:CellTiter-Blue fluorescence, effectively measuring reporter gene-expression per cell.

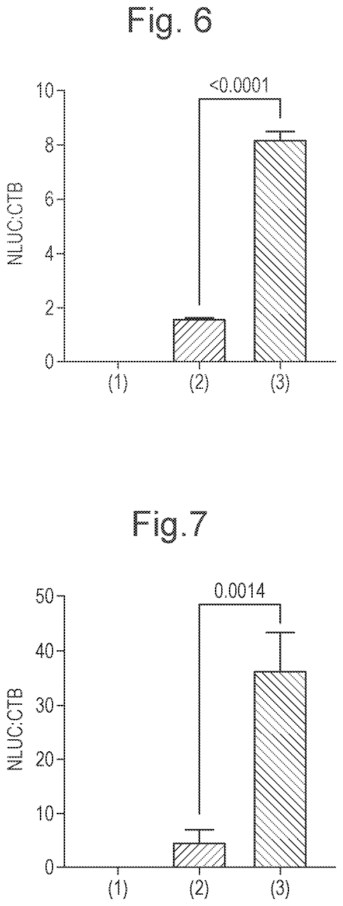

The results of the NanoLuc activity assay are shown in FIGS. 6 (for the untreated control and test polymersome compositions (1) and (2)) and 7 (for the untreated control and test polymersome compositions (3) and (4)). FIG. 6 shows that no NanoLuc expression was observed in the untreated control. For the PEG-PLA polymersomes comprising free pcDNA3.1NL (polymersome formulation (1)), some NanoLuc expression was observed; however, association of pcDNA3.1NL with histone H1 within the polymersome (polymersome formulation (2)) led to an over 4-fold increase in the levels of NanoLuc expression. An analogous conclusion can be drawn from FIG. 7 for the PEG-PDPA polymersomes: in the untreated control, no NanoLuc expression was observed, and whilst NanoLuc expression was observed when the cells were treated both with polymersomes comprising free pDNA cargo (polymersomes (3)) and pDNA associated with nucleic acid-binding protein as cargo (polymersomes (4)), the expression levels are approximately 7-fold higher for the complexed pDNA.

These experiments therefore demonstrate that the polymersome delivery systems of the present invention comprising a cargo comprising both nucleic acid and a nucleic acid-binding protein not only aid the delivery of a nucleic acid cargo to its target cells and improve internalization within the cells, but also ensure an increased efficacy of the nucleic acid cargo is observed at the target site, relative to comparative polymersomes comprising free nucleic acid cargo.

1. A cargo-containing polymersome which comprises:
(a) a polymersome; and
(b) a cargo encapsulated within the polymersome,
wherein said cargo comprises a nucleic acid and a nucleic acid-binding protein.

2. A cargo-containing polymersome according to claim 1, wherein:
(i) said nucleic acid is a DNA and said nucleic acid-binding protein is a DNA-binding protein; or
(ii) said nucleic acid is an RNA and said nucleic acid-binding protein is an RNA-binding protein; or
(i) said nucleic acid is a PNA and said nucleic-acid binding protein is a PNA-binding protein.

3. A cargo-containing polymersome according to claim 2, wherein said nucleic acid is a DNA, and said nucleic acid-binding protein is a histone protein.

4. A cargo-containing polymersome according to claim 3, wherein said nucleic acid is plasmid DNA, and said nucleic acid-binding protein is histone H1.

5. A cargo-containing polymersome according to claim 2, wherein said nucleic acid is an RNA selected from mRNA, siRNA, shRNA, miRNA, or tRNA.

6. A cargo-containing polymersome according to claim 5, wherein said nucleic acid is an mRNA, and said nucleic acid-binding protein is eukaryotic translation initiation factor 4E (EIF4E) or a polyA binding protein.

7. A cargo-containing polymersome according to claim 1, wherein the polymersome comprises a block copolymer having a hydrophobic block and a hydrophilic block.

8. A cargo-containing polymersome according to claim 7, wherein said block copolymer is selected from poly(ethylene glycol)-poly(lactic acid) (PEG-PLA) and poly(ethylene glycol)-poly(2-(diisopropylamino)ethyl methacrylate) (PEG-PDPA).

9. A cargo-containing polymersome according to claim 1, wherein the z-average diameter of the polymersome particles as measured by dynamic light scattering is from 80 to 120 nm.

10. A cargo-containing polymersome according to claim 1, wherein the polymersome is for binding to the surface of a cell and comprises (a) a polymer brush and (b) at least a first ligand type on its external surface, wherein said first ligand type is capable of binding to a first receptor type on said cell surface.

11. A cargo-containing polymersome according to claim 10, wherein the polymersome comprises 2 to 1000 ligands, and preferably from 20 to 200 ligands, of the first ligand type.

12. A cargo-containing polymersome according to claim 10, wherein the polymersome further comprises at least a second ligand type on its external surface, wherein said second ligand type is capable of binding to a second receptor type on said cell surface.

13. A cargo-containing polymersome according to claim 12, wherein the polymersome comprises from 2 to 1000 ligands, and preferably from 20 to 200 ligands, of the second ligand type.

14. A cargo-containing polymersome according to claim 10, wherein the polymersome comprises PEG-PDPA or PEG-PLA and each ligand type on its external surface.

15. A cargo-containing polymersome according to claim 10, wherein each ligand on the external surface of the polymersome is covalently bound to a poly(ethylene glycol) molecule.

16. A cargo-containing polymersome according to claim 1, further comprising a drug encapsulated within the polymersome.

17. A pharmaceutical composition comprising a plurality of the cargo-containing polymersomes according to claim 1, and one or more pharmaceutically acceptable excipients or diluents.

18. (canceled)

19. (canceled)

20. A method of treating cancer, an infectious disease, a brain disorder, an inflammatory or autoimmune disease, atherosclerosis, ischemic heart disease, a liver disorder, a kidney disorder, a disease associated with ageing, or a genetic disease in a human patient, wherein said method comprises administration of a cargo-containing polymersome according to claim 1, to a patient in need thereof.

21. (canceled)

22. A vaccine comprising a cargo-containing polymersome according to claim 1, and one or more pharmaceutically acceptable excipients or diluents.

23. A vaccine according to claim 22, wherein the cargo within the polymersome comprises an mRNA that encodes an antigen, and an mRNA-binding protein.
